(19) United States
(12) Patent Application Publication
(10) Pub. No.: US 2024/0229108 A9
(43) Pub. Date: Jul. 11, 2024

(54) Deoxyribozymes Generating Chemiluminescent Signals

(71) Applicant: USTAV ORGANICKE CHEMIE A BIOCHEMIE AV CR, V.V.I., Praha 6 (CZ)

(72) Inventors: Edward Arthur CURTIS, Fullerton, CA (US); Katerina SVEHLOVA, Praha 7 (CZ); Martin JAKUBEC, Banka (SK); Ondrej LUKSAN, Praha 9 (CZ)

(21) Appl. No.: 18/275,046

(22) PCT Filed: Jan. 12, 2022
(86) PCT No.: PCT/CZ2022/050001

§ 371 Date: Jul. 31, 2023

(30) Foreign Application Priority Data
Feb. 12, 2021 (EP) 21156992.6

(51) Int. Cl. C12Q 1/6823; C12N 15/11

(52) U.S. Cl. CPC C12Q 1/6823; C12N 15/111; C12N 2310/127; C12N 2320/11

(57) ABSTRACT

The present invention relates to self-phosphorylating deoxyribozymes containing or consisting of the nucleotide sequence: 5′-X¹X²X³AX⁴X⁵-R¹-X⁶X⁷X⁸X⁹X¹⁰-bp1-helix1-R²-helix2-bp2-X¹¹TGACX¹²TGGGAX¹³-helix3-X¹⁴-R³-3′ (SEQ ID NO. 1) wherein X¹ is G or T; X² is G or T; X³ is A or C; X⁴ is G or T; X⁵ is A or T; X⁶ is G or T or A; X⁷ is A or C or G or T; X⁸ is A or G or T; X⁹ is T or C or A or G; X¹⁰ is A or T or G; X¹¹ is G or A; X¹² is T or C or G; X¹³ is T or G or C; X¹⁴ is G or A or C or T; R¹ represents a nucleotide sequence containing 3 to 30 nucleotides; R² represents a nucleotide sequence containing 3 to 30 nucleotides; R³ represents a nucleotide sequence containing 0 to 30 nucleotides; bp1 and bp2 are nucleotides selected from G, A, C, T, which together form a base pair; helix1, helix2, and helix3 each independently denotes a nucleotide sequence preferably containing 4 or more nucleotides, wherein helix1, helix2, and helix3 together form a triple helical structure. The deoxyribozymes of the present invention may be used for detection of chemiluminescent substrates, or for detection of analytes (ligands) based on chemiluminescent reaction of the substrates catalyzed by the deoxyribozymes.

### FIELD

The present invention relates to deoxyribozymes and their uses.

### BACKGROUND ART

Once thought to function primarily as a passive carrier of genetic information, RNA and DNA molecules are now known to be capable of a wide range of functions. For example, nucleic acid molecules called ribozymes (RNA) or deoxyribozymes (DNA) can act as catalysts which accelerate reaction rates millions of times relative to the corresponding nonenzymatic reactions. the structures and functions of catalytic nucleic acids can be allosterically modulated by ligands, and this has been utilized to construct sensors made of RNA and DNA (Tang.J. and Breaker,R.R. (1997) Chem Biol, 4, 453-459; Koizumi,M., Soukup,G.A., Kerr,J.N. and Breaker,R.R. (1999) Nat Struct Biol, 6, 1062-1071). Such sensors consist of a binding domain, which binds the ligand, and a signaling domain, which produces a detectable signal only in the presence of the ligand. Early examples used a self-cleaving ribozyme, and reaction products were detected by PAGE. Later experiments used chemically modified RNA substrates containing a fluorophore at one end and a quencher at the other. Ribozyme-catalyzed cleavage separated the fluorophore from the quencher, and generated a fluorescent signal that could be detected in solution and monitored in real time. Several additional signaling domains made of DNA or RNA have recently been described. These include G-quadruplexes with peroxidase activity which can generate colorimetric or chemiluminescent readouts (Kosman,J. and Juskowiak,B. (2011) Anal Chim Acta, 707, 7-17) and RNA aptamers which enhance the fluorescence of ligands to which they bind (Paige,J.S, Wu,K.Y. and Jaffrey,S.R. (2011) Science, 333, 642-646). These domains typically generate signals 10 to 2000-fold higher than background levels. Limitations include the requirement for expensive chemically modified oligonucleotides or fluorophores that are not commercially available.

### DISCLOSURE OF THE INVENTION

The invention relates to a deoxyribozyme which catalyzes a self-phosphorylation reaction in which a phosphate group is transferred from a substrate to the deoxyribozyme. The phosphorylation reaction thus involves cleavage of the phosphate group from the substrate, and its transfer to the deoxyribozyme. The invention further relates to methods of detection of the substrate, or of the deoxyribozyme, or of a ligand such as an oligonucleotide.

The substrate is a compound containing a phosphate functional group and shall be configured so that the cleavage of the phosphate functional group causes a measurable optical signal to be generated. The optical signal may be within the visible light wavelength range, or outside the visible light wavelength range, e.g., in UV or IR wavelengths. Such substrates may be described as chemiluminescent.

The chemiluminescent substrate is preferably a compound of the group of phosphate-stabilized 1,2-dioxetanes. Such substrates are described, e.g., in Bronstein,I., Edwards,B. and Voyta,J.C. (1989) J Biolumin Chemilumin, 4, 99-111; Trayhurn,P., Thomas,M.E., Duncan,J.S., Black,D., Beattie,J.H. and Rayner,D.V. (1995) Biochem Soc Trans, 23, 494S. The compound disclosed in the cited prior art as phosphate-stabilized 1,2-dioxetanes or as chemiluminescent substrates are incorporated herein as examples of chemiluminescent substrates.

1,2-dioxetanes are 4-member ring peroxides which decompose (in the case of our substrates, after cleavage of the stabilizing phosphate group) to form products which are in an excited electronic state. These products may decompose further and emit light.

An example of a chemiluminescent substrate is disodium 2-chloro-5-(4-methoxyspiro{1,2-dioxetane-3,2′-(5′-chloro)tricyclo[3.3.1.1^{3,7}]decan}-4-yl)phenyl phosphate (commercially available under the trade name CDP-Star™). The CDP-Star substrate is known as a substrate of alkaline phosphatase for use in detection of alkaline phosphatase or alkaline phosphatase conjugates, and for detection of non-radioactively labeled nucleic acids in Southern blots, Northern blots, dot blots, colony or plaque hybridizations, gel shift assays. Upon dephosphorylation, the CDP-Star substrate forms meta-stable dioetane phenolate anion which decomposes and emits light at 466 nm.

Another example of a chemiluminescent substrate is disodium 3-(4-methoxyspiro {1,2-dioxetane-3,2′-(5′-chloro)tricyclo[3.3.1.13.71]decan}-4-yl) phenyl phosphate (commercially available, under the trade name CSPD™). The chemical structure is similar to CDP-Star, and it is used for similar applications.

The deoxyribozyme of the present invention contains or consists of the following nucleotide sequence:

(SEQ ID NO. 1)
5′-X¹X²X³AX⁴X⁵S-R¹-X⁶X⁷X⁸X⁹X¹⁰-bp1-helix1-R²-
helix2-bp2-X¹¹TGACX¹²TGGGAX¹³-helix3-X¹⁴-
R³-3′

wherein
X¹ is G or T; Z² is G or T; X³ is A or C; X⁴ is G or T; X⁵ is A or T; X⁶ is G or T or A; X⁷ is A or C or G or T; X⁸ is A or G or T; X⁹ is T or C or A or G; X¹⁰ is A or T or G; X¹¹ is G or A; X¹² is T or C or G; X¹³ is T or G or C; X¹⁴ is G or A or C or T;
R¹ represents a nucleotide sequence containing 3 to 30 nucleotides;
R² represents a nucleotide sequence containing 3 to 30 nucleotides;
R³ represents a nucleotide sequence containing 0 to 30 nucleotides;
bp1 and bp2 are nucleotides selected from G, A, C, T, which together form a base pair;
helix1, helix2, and helix3 each independently denotes a nucleotide sequence preferably containing 4 or more nucleotides, helix1, helix2 and helix3 together form a triple helical structure.

In some embodiments, the deoxyribozyme of the present invention contains or consists of the following nucleotide sequence:

(SEQ ID NO. 1)
5′-X¹X²X³AX⁴AX⁵-R¹-X⁶X⁷X⁸X⁹X¹⁰-bp1-helix1-R²-
helix2-bp2-X¹¹TGACX¹²TGGGAX¹³-helix3-X¹⁴-
R³-3′

wherein
X¹ is G; X² is G; X³ is A or C; X⁴ is G; X⁵ is A or T; X⁶ is G; X⁷ is A; X⁸ is A, X⁹ is T or C; X¹⁰ is A; X¹¹ is G; X¹² is T or C; X¹³ is T; X¹⁴ is G or A or C or T;
and wherein one or more, preferably one or two, of X¹-X¹⁴ are replaced by the following nucleotides:
X¹=T; X²=T; X⁴=T; X⁶=T or A; X⁷=C or G or T; X⁸=G or T; X⁹=A or G; X¹⁰=T or G; X¹¹=A; X¹²=G; X¹³=G or C;
R¹ represents a nucleotide sequence containing 3 to 30 nucleotides;
R² represents a nucleotide sequence containing 3 to 30 nucleotides;
R³ represents a nucleotide sequence containing 0 to 30 nucleotides;
bp1 and bp2 are nucleotides selected from G, A, C, T, which together form a base pair;
helix1, helix2 and helix3 each independently denotes a nucleotide sequence preferably containing 4 or more nucleotides, wherein helix1, helix2, and helix3 together form a triple helical structure.

In some embodiments, the deoxyribozyme of the present invention contains or consists of the following nucleotide sequence:

(SEQ ID NO. 2)
5′-GGX³AGX⁵-R¹-GAAX⁹A-bp1-helix1-R²-helix2-
bp2-GTGACX¹²TGGGAT-helix3-X¹⁴-R³-3′

wherein
X³ is A or C; X⁵ is A or T; X⁹ is T or C; X¹² is T or C; X¹⁴ is G or A or C or T;
R¹ represents a nucleotide sequence containing 3 to 30 nucleotides;
R² represents a nucleotide sequence containing 3 to 30 nucleotides;
R³ represents a nucleotide sequence containing 0 to 30 nucleotides;
bp1 and bp2 are nucleotides selected from G, A, C, T, which together form a base pair;
helix1, helix2, and helix3 each independently denotes a nucleotide sequence preferably containing 4 or more nucleotides, wherein helix1, helix2, and helix3 together form a triple helical structure.

In some embodiments, the deoxyribozyme contains or consists of the following nucleotide sequence:
5′-GGAAGA-R¹-GAATA-bp1-helix1-R²-helix2-bp2-GTGACTTGGGAT-helix3-G-R³-3′ (SEQ ID NO. 3), wherein
R¹ represents a nucleotide sequence containing 3 to 30 nucleotides;
R² represents a nucleotide sequence containing 3 to 30 nucleotides;
R³ represents a nucleotide sequence containing 0 to 30 nucleotides;
bp1 and bp2 are nucleotides selected from G, A, C, T, which together form a base pair;
helix1, helix2, and helix3 each independently denotes a nucleotide sequence preferably containing 4 or more nucleotides, wherein helix1, helix2, and helix3 together form a triple helical structure.

Generally, nucleotides are selected from adenine nucleotide (A), cytosine nucleotide (C), guanine nucleotide (G), and thymine nucleotide (T). A nucleotide generally contains a base (adenine or cytosine or guanine or thymine) covalently linked to a deoxyribose which is in turn covalently linked to a phosphate.

Preferably, R¹ contains 3 to 30 nucleotides, more preferably 3 to 4 nucleotides.

In some embodiments, R¹ is a poly-A sequence.

Preferably, R² contains 3 to 30, more preferably 3 to 10 nucleotides.

In some embodiments, R² is a poly-A sequence. In some embodiments, R² is or contains an aptamer or a sequence complementary to an aptamer.

Preferably, R³ contains 3 to 30, more preferably 3 to 10 nucleotides.

In some embodiments, R³ is a poly-A sequence. In some embodiments, R³ is or contains an aptamer or a sequence complementary to an aptamer.

In some embodiments, when R² is or contains an aptamer, then R³ is or contains a sequence complementary to the aptamer, and when R³ is or contains an aptamer, then R² is or contains a sequence complementary to the aptamer.

An aptamer is an oligonucleotide sequence that binds to a specific target molecule or target sequence (target molecules and target sequences fall into the group called herein more generally “ligands”). Many aptamer sequences for specific target molecules are known in the art (for example in: Gold,L., Polisky,B., Uhlenbeck,O. and Yarus,M. (1995) Annu Rev Biochem, 64, 763-797; Cho,E.J., Lee,J.W. and Ellington,A.D. (2009) Annu Rev Anal Chem, 2, 241-264). An aptamer for a target sequence is a complementary sequence.

The base pair bp1 and bp2 is preferably formed by canonical T-A, A-T, C-G, and G-C base pairs, more preferably by T-A and A-T base pairs.

The triple helical structure is preferably formed by combinations of canonical C-G:G, T-A:A, and T-A:T base triples, more preferably by C-G:G base triples.

Preferably, each of the helixes 1-3 has the length of 4-10 nucleotides, more preferably 4-7 nucleotides.

In some embodiments, helix 1, helix 2, helix 3 is CCCC, GGGG, GGGG or CCCT, AGGG, GGGT or CTCC, GGAG, GAGG or CCCT, AGGG, GGGA.

In one preferred embodiment, helix1 has the sequence of CCCC, helix 2 has the sequence GGGG, and helix 3 has the sequence GGGG.

Partially disrupted triples can also occur at some positions. These have the ability to form either the Watson-Crick part of the base triple (represented by “-”) or the Hoogsteen part of the base triple (represented by “:”).

When each of the helices 1-3 has the length of 4 nucleotides, they form four triples.

The first triple (positions 13-20-34 in the minimized catalytic core; see FIG. 4 for the numbering scheme) may be selected from A G:G, T G:G, A A:T, G G:G, A A:A, G A:A, G A:T, and C A:A, preferably A G:G and T G:G.

The second triple (positions 14-19-35 in the minimized catalytic core; see FIG. 4 for the numbering scheme) may be selected from C-G T, G A:A, C-G A, A G:G, T G:G, A A:A, G A:T, A A:T, C A:A, and G G:G, preferably C-G T and G A:A.

The third triple (positions 15-18-36 in the minimized catalytic core; see FIG. 4 for the numbering scheme) may be selected from G G:G, T G:G, A A:T, G A:T, C-G T, A A:A, A G:G, C-G A, C A:A, and T-A G, preferably G G:G and T G:G.

The fourth triple (positions 16-17-37 in the minimized catalytic core; see FIG. 4 for the numbering scheme) may be selected from C A:T, A A:T, A G:G, A-T A, G A:T, T G:G, C-G T, A A:A, C-G A, C A:A, G-C A, T G T, G G:G, T-A G, G A:A, G T A, G G C, and C A G, preferably C A:T and A A:T.

Preferably, the deoxyribozyme contains 45 to 160 nucleotides, more preferably 45 to 110 nucleotides in total, even more preferably 80 to 110 nucleotides.

The invention thus provides a deoxyribozyme that phosphorylates itself in the presence of a phosphate group-containing substrate, such as phosphate-stabilized 1,2-dioxetane substrates, to generate an optical signal. By “deoxyribozyme”, it is meant a DNA oligonucleotide which catalyzes a chemical reaction. This meaning includes DNA oligonucleotides with chemical modifications. Deoxyribozymes of the present invention were developed with the aid of a method of artificial evolution called in vitro selection. Catalytic oligonucleotides isolated using in vitro selection typically contain a catalytic core, usually consisting of stems forced by A-T, T-A, G-C, and C-G base pairs, interspersed with sequence elements not required for function.

The catalytic core of the deoxyribozyme described herein has the following features:
L1 (i.e., the sequence X¹X²X³AX⁴X⁵) occurs at the 5′ end of the deoxyribozyme. The consensus sequence of L1 is GGAAGA. Of the 50 most abundant deoxyribozyme variants identified (which make up 5.1 million of the 7.1 million total reads generated using high-throughput sequencing), 94.2% contain GGAAGA here. Other possibilities include GGCAGA (3.2% of these reads), and GGAAGT (2.6% of these reads).
L1 is connected to L2 (i.e., the sequence X⁶X⁷X⁸X⁹X¹⁰) by a spacer R¹. The spacer may contain any combination of nucleotides selected from A, C, G, and T. A typical length of the spacer is from 3 to 30 nucleotides. 23,186 different variants of R¹were identified among the 135,000 different deoxyribozyme variants we identified (in this case R¹ contained 26 nucleotides).
The consensus sequence of L2 (i.e., the sequence X⁶X⁷X⁸X⁹X¹⁰) is GAATA. Of the 50 most abundant deoxyribozyme variants identified (which make up 5.1 million of the 7.1 million total reads), 83.2% contain GAATA here. Other possibilities include GCATA (8.4% of these reads), GAATT (5.7% of these reads), and GTATA (0.8% of these reads).
L2 is connected at its 3′ end to a base pair which may consist of T-A, T, C-G, or G-C.
The 3′ end of the 5′ position in the base pair is connected to a triple helix.
The triple helix formed from helix1, helix2, and helix3 may contain various combinations of canonical C-G:G, T-A:A, and T-A:T triples. Optionally other nucleotide triples may be present, as long as the triple-helical structure is maintained.
The triple helix is capped by a loop R² with a sequence N₃₋₃₀ (N=A, C, G or T). 18,024 different variants of R² were identified among the 135,000 different deoxyribozyme variants we identified (in this case R² contained 18 nucleotides).
L3 (i.e., the sequence X¹¹TGACX¹²TGGGAX¹³, SEQ ID NO. 241) connects the 3′ end of helix2 to helix3. The consensus sequence of L3 is GTGACTTGGGAT (SEQ ID NO. 242). Of the 50 most abundant deoxyribozyme variants identified (which make up 5.1 million of the 7.1 million total reads), 100% contain GTGACTTGGGAT (SEQ ID NO. 242) here.
The 3′ end of the deoxyribozyme contains a single nucleotide overhang (X¹⁴). Of the 50 most abundant deoxyribozymes variants identified (which make up 5.1 million of the 7.1 million total reads), 86% contain G here. Other possibilities include T (12% of these reads), and A (2% of these reads).

This overhang can be extended by the extension R³ with the sequence N₀₋₃₀ (N=A, C, G, or T).

The deoxyribozyme of the present invention, upon contact with a substrate, can cause the generation of an optical signal. The optical signal is generated by dephosphorylation of the substrate by the deoxyribozyme. This triggers decomposition of the substrate and, in case of the CDP-Star™ and CSPD™ substrates, production of blue light with a wavelength of ˜466 nm by a CIEEL mechanism. The deoxyribozyme can be configured so that it is inactive (inactive state) in the absence of a ligand, but undergoes an allosteric change to the catalytically active structure (active state) in the presence of the ligand. In this way the deoxyribozyme can be used to detect specific ligands. The substrate is as defined herein above.

The present invention further relates to a method of in vitro detection of a chemiluminescent substrate, containing a phosphate functional group, in a sample, containing the steps of:
bringing the sample into contact with the deoxyribozyme of the present invention;
causing the generation of an optical signal by cleavage of the phosphate functional group from the substrate present in the sample by self-phosphorylation of the deoxyribozyme;
detecting the generated optical signal;
optionally detecting the amount of the generated optical signal.

In this method, it is advantageous when R¹ and R² are not mutually complementary.

In this method, it is advantageous when R¹ and R³ are not mutually complementary.

In this method, it is advantageous when R² and R³ are not mutually complementary.

The present invention also relates to a method of in vitro detection of the deoxyribozyme in a sample, containing the steps of:
bringing the sample into contact with a chemiluminescent substrate containing a phosphate functional group;
causing the generation of an optical signal by cleavage of the phosphate functional group from the substrate by self-phosphorylation of the deoxyribozyme present in the sample;
detecting the generated optical signal;
optionally detecting the amount of the generated optical signal.

The step of causing the generation of the optical signal typically includes providing suitable reaction conditions for the self-phosphorylation reaction of the deoxyribozyme. Such reaction conditions may include, for example, metal ions in the buffer, including potassium, zinc, cerium, lead (e.g., up to 200 mM KCl, 1 mM ZnCl₂, 1 μM Ce(SO₄)₂, and 0.1 μM PbCl₂), pH (e.g. 7-8), buffer (e.g. HEPES), temperature (e.g. 20-30° C.), deoxyribozyme concentration (e.g. 1-50 μM or 0.1-10 μM), substrate concentration (e.g. 0.001-1 mM, preferably 0.01-100 μM) and/or ligand concentration (e.g. 1 to 100 μM).

In some preferred embodiments, the deoxyribozyme is also active in a simplified buffer containing HEPES, zinc, and potassium. In such a buffer, light is most efficiently generated over a narrow range of zinc concentrations (between around 0.3 mM and 1.5 mM) and pH values (between around pH 6.9 and 7.7). Potassium slightly increases light production, but is inhibitory at concentrations above about 200 mM. Light production can also be increased by reducing the substrate concentration (˜62.5 μM is optimal) and increasing the deoxyribozyme concentration (about 30 μM is optimal). Rate enhancements of light production (defined as the amount of light produced in the presence of deoxyribozyme divided by the amount of light produced in the absence of deoxyribozyme) can also be increased by using optimized concentrations of buffer components either individually (e.g. up to 200 mM KCl, 1 mM CDP-Star, 30 μM deoxyribozyme, 650 μM ZnCl₂, 15% DMSO, and 50 mM HEPES, pH 7.4, column 4 in FIG. 8F) or in combination (e.g., 20 mM KCl, 62.5 μM CDP-Star, 30 μM deoxyribozyme, 650 μM ZnCl₂, 15% DMSO, and 50 mM. HEPES, pH 7.4, column 8 M FIG. 8F).

The present invention further relates to a method of in vitro detection of a ligand in a sample, containing the steps of:
bringing the sample into contact with the deoxyribozyme of the present invention and with a substrate containing a phosphate functional group and capable of generating an optical signal upon cleavage of the phosphate functional group; wherein the deoxyribozyme is in an inactive state (induced by an inhibitory helix formed by base pairing between a sequence inserted into R² and R³) and wherein R³ contains an aptamer for the ligand so that when the ligand binds to the aptamer, the deoxyribozyme is converted into an active state,;
causing the generation of an optical signal by cleavage of the phosphate functional group from the substrate present in the sample by self-phosphorylation of the deoxyribozyme in the active state;
detecting the generated optical signal;
optionally detecting the amount of the generated optical signal.

The ligand may be, for example, an oligonucleotide, a small molecule (e.g., molecular weight up to 2000 g/mol), a metal ion, a protein, or a lipid. The ligand is in fact an analyte detected in a sample.

The ligand binds to the aptamer, thus changing the conformation of the deoxyribozyme from the inactive state to the active state, and the deoxyribozyme in the active state then self-phosphorylates, thus cleaving the phosphate group from the substrate. This triggers a decomposition reaction and the production of blue light by a CIEEL mechanism.

For example, when the ligand is an oligonucleotide, the R² in the deoxyribozyme may be or contain the sequence or part of the sequence of the ligand (oligonucleotide), and the R³ in the doxyribozyme may be or contain a sequence complementary to the sequence or to part of the sequence of the ligand. The oligonucleotide binds to R³ by Watson-Crick pairing. At sufficiently high concentrations, this prevents formation of the inhibitory interaction between R² and R³. When the oligonucleotide is bound to R³, the deoxyribozyme is catalytically active and transfers the phosphate group from the substrate to the deoxyribozyme. This triggers a decomposition reaction and the production of blue light by a CIEEL mechanism.

Also when the ligand is a small molecule or a metal ion, or a protein, or a lipid, binding of the ligand to an aptamer suitable for the ligand causes conformational changes in the deoxyribozyme leading to its self-phosphorylation. For example, a sequence complementary to the aptamer can be inserted into R² and the aptamer can be inserted into R³. In the absence of the ligand of the aptamer, an inhibitory interaction will form between R² and R³ by Watson-Crick pairing. In the presence of sufficiently high concentrations of the ligand, however, the aptamer will bind the ligand rather than R². This will prevent formation of the inhibitory interaction between R² and R³. When the ligand is bound to the aptamer, the deoxyribozyme is catalytically active and transfers the phosphate group from the substrate to the deoxyribozyme. This triggers a decomposition reaction and the production of blue light by a CIEEL mechanism.

The step of causing the generation of the optical signal typically includes providing suitable reaction conditions for the self-phosphorylation reaction of the deoxyribozyme. Such reaction conditions may include, for example, metal ions in the buffer, including potassium, zinc, cerium, lead (e.g., 200 mM KCl, 1 mM ZnCl₂, 1 μM Ce(SO₄)₂, and 0.1 μM PbCl₂), pH (e.g. 7-8), buffer (e.g. HEPES), temperature (e.g. 20-30° C.), deoxyribozyme concentration (e.g. 0.1-10 μM), and/or substrate concentration (e.g. 0.001-1 mM), and ligand concentration (e.g. 1 to 100 μM).

Light generated by the deoxyribozyme can be detected using an instrument such as a plate reader, e.g. Tecan Spark plate reader.

Generally, the detection of the light may be qualitative or quantitative. Quantitative detection contains an additional step of determining the amount of the optical signal generated.

The invention also relates to the use of the deoxyribozyme of the present invention as a signaling component of molecular devices in applications such as biosensing and molecular computing. Molecular devices may include molecular sensors or molecular switches, such as deoxyribozymes that only generate light in the presence of a specific ligand such as ATP. Suitable applications of such molecular devices include diagnostics, high-throughput screens, and molecular computing. The deoxyribozyme of the present invention is used in the molecular device as the signaling component causing the generation of an optical signal which indicates the presence of a ligand (e.g., a metal ion, a small molecule, a protein, an oligonucleotide, a lipid), and optionally the molecular device is configured to perform an action upon generation of the said signal.

A second object of the invention relates to the use of this deoxyribozyme in molecular devices that generate a chemiluminescent signal. An example of such a device is an allosterically regulated sensor in which the deoxyribozyme described here (the signaling domain) is covalently linked to an aptamer (the binding domain) that binds a small molecule, oligonucleotide, or protein with high affinity and specificity. An example of such a sensor is shown in FIG. 7 and Example 15. The deoxyribozyme contains an oligonucleotide binding site, and the amount of light produced is correlated with the concentration of the oligonucleotide in the buffer from 100 nM to 10 μM. Such a sensor can be constructed to detect any ligand, based on the information available in the art and based on the knowledge of a person skilled in the art.

### FIGURES

FIG. 1: Reaction scheme of deoxyribozyme-catalyzed light production using the CDP-Star™ substrate.

FIG. 2: Schematic diagram of the method used to develop deoxyribozymes that phosphorylate themselves in the presence of the chemiluminescent substrate as described in Example 1.

FIG. 3: Light production of a deoxyribozyme in the presence of the chemiluminescent CDP-Star™ substrate (see also Example 4).

FIG. 4: Minimized catalytic core and preferred nucleotides at unpaired positions. (A) Deoxyribozyme before minimization (see also Example 2). (B) Deoxyribozyme after minimization (see also Example 7). Solid horizontal lines hydrogen bonds in base pairs in the Watson-Crick duplex of the triple helix; dashed horizontal lines=hydrogen bonds formed by the third strand of the triple helix. Note that only positions in the minimized catalytic core of the deoxyribozyme (shown in capital letters) are numbered. (C) Effects of mutations in unpaired positions. Only mutations with enrichment values≥3.1×10⁻³ (frequency in the evolved pool divided by frequency in the starting pool) are shown. Percent activity is relative to SEQ ID NO:15 (shown in panel B and indicated by a dash in the graph). See also Example 9 and Table 2.

FIG. 5: Secondary structure model and analysis of base triples. (A) Secondary structure model. (B) Triple-mutant cycle consistent with the 15-18-36 base triple. See also Example 8. (C) Activity of canonical, partially disrupted (“partial”), and fully disrupted (“other”) triples. Each triple was inserted into SEQ ID NO:15 (shown in panel A) at either 13-20-34, 14-19-35, 15-18-36 or 16-17-37; the average activity of these four mutants is indicated by each bar. See also Example 11 and Table 4.

FIG. 6: (A) Secondary structure model showing positions (R¹, R², and R³) at which spacers with different lengths and sequences can be inserted. (B) Activity of deoxyribozymes containing randomized spacers of different lengths at R¹, R², or R³. N indicates a position containing 25% A, 25% C, 25% G, and 25% T. See also Example 13. (C) Activity of deoxyribozymes in which randomly chosen spacers of different lengths and sequences were inserted into R¹, R², and/or R³. Five different examples were tested for each architecture. Sec also Example 14.

FIG. 7: Deoxyribozyme sensor that produces light in the presence of a specific oligonucleotide, Example 5. (A) Design of the sensor. Left: deoxyribozyme before engineering. Middle: engineered deoxyribozyme sensor in the OFF conformation. 1 and 1c represent complementary sequences. Right: engineered deoxyribozyme sensor in the ON conformation in which the ligand (in this case an oligonucleotide) is bound to 1c by base pairing. (B) Sensitivity of the sensor. Production of light as a function of DNA concentration for an oligonucleotide complementary to the deoxyribozyme sensor. (C) Specificity of the sensor. Light production of five deoxyribozyme sensors (Example 15) in the presence of either an oligonucleotide complementary to the sensor (i.e. sensor 1 with target 1) or oligonucleotides not complementary to the sensor (i.e. sensor 1 with targets 2, 3, 4, or 5). Black squares indicate pairs for which the rate enhancement (light produced in the presence of target divided by light produced in the absence of target) exceeds 5-fold.

FIG. 8: Effect of reaction conditions on deoxyribozyme-catalyzed light production, Example 16.

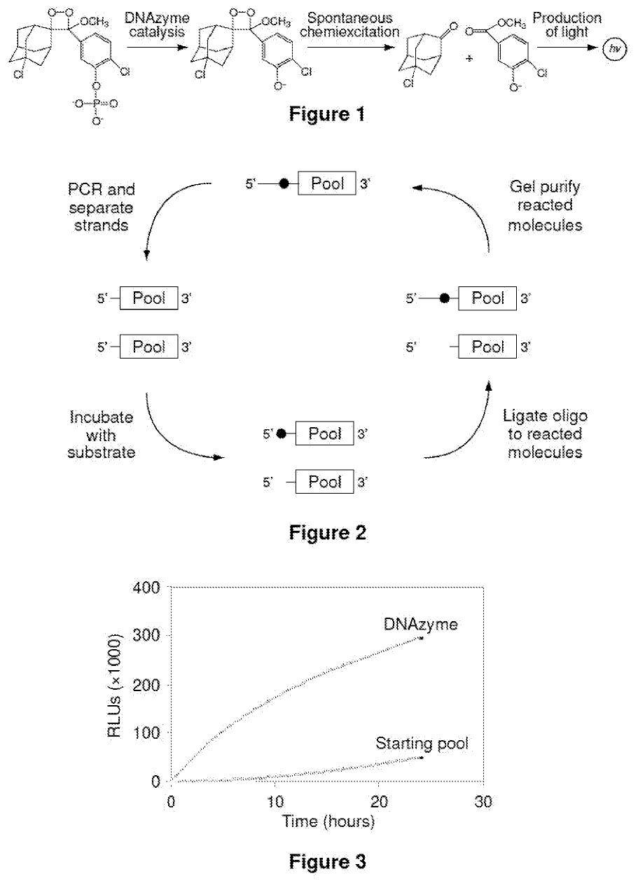

### EXAMPLES

### Example 1: Procedure for Selecting Light-Producing Deoxyribozymes

### Initial Selection

A single-stranded pool with the sequence GGAAGAGATGGCGACN₇₀AGCTGATCCTGATGG (SEQ ID NO. 4) was ordered from IDT and purified by PAGE. 10¹⁶ different sequences from this pool were mixed with the blocking oligonucleotide BO1 (CCATCAGGATCAGCT, SEQ ID NO. 5) in water, heated at 65° C. for 2 minutes and cooled at RT for 5 minutes. They were then incubated for 24 hours (round 1-6) or 10 minutes (round 7-9) with the substrate CDP-Star (Roche) as follows: 1 μM DNA pool, 1.5 μM blocking oligonucleotide BO1, 1× selection buffer (50 mM HEPES pH 7.4, 200 mM KCl, 1 mM ZnCl₂, 1 μM Ce(SO₄)₂, 0.1 μM PbCl₂), and 1 mM CDP-Star DNA was then precipitated in ethanol, and ligated to a short oligonucleotide with the sequence. GAATTCTAATACGACTCACTATA (SEQ ID NO. 6) using a splint-ligation method, T4 DNA ligase (Jena Bioscience), and a splint oligonucleotide with the sequence GTCGCCATCTCTTCCTATAGTGAGTCGTATTAG (SEQ ID NO. 7). All oligonucleotides were kept at a concentration of 2.5 μM. Molecules were then separated by gel shift using 6% Urea-PAGE, and molecules of the size 120 nt (corresponding to ligated pool members) were excised from the gel, eluted, and precipitated with ethanol. These molecules were then amplified by PCR using Tag Polymerase (Jena Bioscience) and the FWD1 (GAATTCTAATACGACTCACTATA, SEQ ID NO. 6) and REV1 (CCATCAGGATCAGCT, SEQ ID NO. 5) primers. The FWD1 primer contained a single RNA linkage at its 3′ end so the reaction site of the deoxyribozyme could be regenerated. After another ethanol precipitation, the sense strands of the double-stranded molecules were base hydrolyzed, and the two strands were separated using 6% Urea-PAGE to regenerate the single-stranded DNA pool.

### Re-selection

A library was generated by randomly mutagenizing the most active deoxyribozyme isolated in the original selection:
GGAAGAGATGGCGACGACACAGGGACGATGCCGAATATCCTCAGTGCGCAGGGCCGCAG GGGGGAGTGACTTGGGATGGGGGGTCAGCTGATCCTGATGG (SEQ ID NO. 8) at a rate of 21% per position. The sequence of this library was GGAAGAGATGGCGACGACACAGGGACGATGCCGAATATCCTCAGTGCCGAG GGCCGCAGGGGGGAGTGACTTGGGATGGGGGGTCCACTAATGATCTGCCCGATG (SEQ ID NO. 9) (mutagenized positions are indicated in bold). This library was ordered from IDT and purified by PAGE. 10¹⁴ different sequences from this pool were mixed with the blocking oligonucleotide BO2 (CATCGGGCAGATCATTAGTG, SEQ ID NO. 10) in water, heated at 65° C. for 2 minutes and cooled at RT for 5 minutes. The mixture was then incubated for 10 minutes (round 1-3) or 1 minute (round 4-7) with CDP-Star at the following final concentrations: 1 μM DNA pool, 1.5 μM blocking oligonucleotide (BO2), 1× selection buffer, and 1 mM CDP-Star. DNA was then ethanol precipitated, and ligated to a short oligonucleotide with the sequence ACCGCTCAGGTGTAGTATCA (SEQ ID NO. 11) using a splint-ligation method, T4 DNA ligase (Jena Bioscience GmbH), and a splint oligonucleotide with the sequence GTCGCCATCTCTTCCTGATACTACACCTGAGCGGT (SEQ ID NO. 12). All oligonucleotides were kept at a concentration of 2.5 μM. Molecules were then separated by gel shift using 6% Urea-PAGE, and molecules of the size of 125 nt (corresponding to ligated pool members) were excised from the gel, eluted, and precipitated with ethanol. These molecules were then amplified by PCR using Q5 Hot Start Polymerase (NEB) and the FWD2 (ACCGCTCAGGTGTAGTATCA, SEQ ID NO. 13) and REV2 (CATCGGGCAGATCATTAGTG, SEQ ID NO. 10) primers. The FWD2 primer contained a single RNA linkage at its 3′ end so the reaction site of the deoxyribozyme could be regenerated, and the REV2 primer contained a 5′ phosphate group so the reverse strand could be selectively degraded. The reactions were then purified using the NucleoSpin Gel and PCR Clean-up kit (Macharey-Nagel), and the reverse strand was removed by digestion with λ-exonuclease (NEB). After a second purification with the NucleoSpin Gel and PCR Clean-up kit, the remaining sense strands were base hydrolyzed, and purified using 6% Urea-PAGE to regenerate single-stranded DNA pool.

### Example 2: Consensus Sequence Based on an Alignment of 135,000 Light-Producing Deoxyribozyme Variants Isolated by In Vitro Selection

After 6 rounds of the reselection the evolved pool was characterized by high-throughput sequencing. The analysis was performed using the Illumina HiSeq System (2×150 bp, paired-end; NGSelect Amplicon product by Eurofins Genomics). Raw reads were processed with the cutadapt tool to remove adapter and primer sequences, perform quality trimming, and filter low quality reads. Paired-end reads were oriented, merged with the program fastq-join, and aligned using Clustal Omega. Data quality was evaluated using the FastQC tool. The secondary structure of the deoxyribozyme was predicted using comparative sequence analysis, which included frequency calculations of all di- and trinucleotide combinations and mutual information analysis (Gutell,R.R., Power,A., Hertz,G.Z., Putz,E.J. and Stormo,G.D. (1992). Nuc Acids Res, 20, 5785-57951) using in-house scripts. Analysis of conservation led to the identification of the consensus sequence 5′ GGAAGA-(26 variable nucleotides)-GAATATCCCC-(18 variable nucleotides)-GGGGAGTGACTTGGGATGGGGG 3 (SEQ ID NO. 14). Detailed information about all tools used is listed in Table 1.

TABLE 1
Tools used for data analysis

| Tool | Version | Source |
|---|---|---|
| cutadapt | 1.15 | https://cutadapt.readthedocs.io/en/stable/ |
| fastq-join | 1.3.1 | https://github.com/brwnj/fastq-join |
| Clustal Omega | 1.2.4 | https://www.ebi.ac.uk/Tools/msa/clustalo/ |
| FastQC | 0.11.5 | https://www.bioinformatics.babraham.ac.uk/ |
| | | projects/fastqc/ |
| ViennaRNA | 2.4.12 | https://www.tbi.univie.ac.at/RNA/ |

### Example 3: Measuring Deoxyribozyme Catalytic Activity using a Ligation Assay

Oligonucleotides were ordered from Sigma-Aldrich and purified by PAGE or HPLC prior to use. Each deoxyribozyme was mixed with either water alone or water and the appropriate blocking oligonucleotide (indicated in Examples 5 and 6), heated at 65° C. for 2 minutes, and cooled at RT for 10 minutes. After that, 5× Selection Buffer and CDP-Star™ were added. Final concentrations were 1 μM deoxyribozyme, 1.5 μM blocking oligonucleotide (when needed), 1× Selection Buffer (50 mM HEPES pH 7.4, 200 mM KCl, 1 mM ZnCl₂, 1 M Ce(SO₄)₂, 0.1 μM PbCl₂), and 1 mM CDP-Star™. Reactions were incubated for various times in the dark at RT and ethanol-precipitated. Deoxyribozymes were then ligated to a short oligonucleotide as described in Example 1. Reacted and unreacted molecules were then separated on 6% PAGE gels, and the amount of ligated product was determined using ImageQuant TL software (GE Healthcare LifeSciences).

### Example 4: Generation of a Chemiluminescent Signal using the Deoxyribozyme

Sequences of the isolated deoxyribozymes were ordered from Sigma-Aldrich and purified by PAGE or HPLC prior to use. Deoxyribozymes were mixed with either water or water and the appropriate blocking oligonucleotide, heated at 65° C. for 2 minutes, and cooled at RT for 10 minutes. After that, 5× Selection Buffer was added, and samples were transferred to a white half-area 96 well plate (Corning). CDP-Star™ was added, and chemiluminescence was measured for 1 to 24 hours using a Tecan Spark plate reader (Tecan Group). Final concentrations were 1 μM deoxyribozyme, 1.5 μM blocking oligonucleotide (when necessary), 1× Selection Buffer (50 mM HEPES pH 7.4, 200 mM KCl, 1 mM ZnCl₂, 1 μM Ce(SO₄)₂, 0.1 μM PbCl₂), and 1 mM CDP-Star™.

### Example 5: Catalytic Activity of Deoxyribozymes from the Initial Selection

Deoxyribozymes from the initial selection were tested as described in Example 4, except using 0.25 mM rather than 1 mM CDP-Star™ substrate. The most active variant was

(SEQ ID NO. 8)
GGAAGAGATGGCGACGACACAGGGACGATGCCGAATATCC
TCAGTGCGCAGGGCCGCAGGGGGGAGTGACTTGGGATGGG
GGGTCAGCTGATCCTGATGG,

and it generated light with a rate enhancement of 39.6-fold relative to the reaction in the absence of deoxyribozyme. The blocking oligonucleotide used was REV1 (CCATCAGGATCAGCT, SEQ ID NO. 5).

### Example 6: Catalytic Activity of Deoxyribozymes from the Reselection

The ten most abundant deoxyribozymes in the reselection were tested as described in Example 4, except using 0.25 mM rather than 1 mM CDP-Star™ substrate. The most abundant deoxyribozyme was

(SEQ ID NO. 15)
GGAAGATATGGCGCGAACAAATGGACAATGCCGAATATCC
CCCGCACGCAGGGCAACAAGGGGGAGTGACTTGGGATGGG
GGCTGCACTAATGATCTGCCCGATG

and it generated light with a rate enhancement of 97.9-fold relative to the reaction in the absence of deoxyribozyme. The blocking oligo used was REV2 (CATCGGGCAGATCATTAGTG, SEQ ID NO. 10).

The second most abundant deoxyribozyme was

(SEQ ID NO. 16)
GGAAGAAATGACGAGGCCACACGGACGAGGCTGAATATCC
TCACTGCGGTGTGCCCCAGGGAGGAGTGACTTGGGATGGT
GGGTTCACTAATGATCTGCCCGATG

and it generated light with a rate enhancement of 130.4-fold relative to the reaction in the absence of deoxyribozyme. The blocking oligo used was REV2 (CATCGGGCAGATCATTAGTG, SEQ ID NO. 10).

The third most abundant deoxyribozyme was

(SEQ ID NO. 17)
GGAAGATAGTGTGTCGATAAAGGGACAATGCTGAATTTCC
TCCCTGAGCOGGCGCGTAGGGGGGAGTGACTTGGGATGGG
GGGTGCACTAATGATCTGCCCGATG

and it generated light with a rate enhancement of 187-fold relative to the reaction in the absence of deoxyribozyme. The blocking oligo used was REV2 (CATCGGGCAGATCATTAGTG, SEQ ID NO. 10).

The fourth abundant deoxyribozyme was

(SEQ ID NO. 18)
GGAAGACTTGACGAGCGCACAGCGCTTGTTCAGAATATCA
CCCGTCATGTGTTCGTGAAGGGGGAGTGACTTGGGATGGG
GGTTCCACTAATGATCTGCCCGATG

and it generated light with a rate enhancement of 129.2-fold relative to the reaction in the absence of deoxyribozyme. The blocking oligo used was REV2 (CATCGGGCAGATCATTAGTG, SEQ ID NO. 10).

The fifth most abundant deoxyribozyme was

(SEQ ID NO. 19)
GGAAGAAATGGTGCGACACAGGGGCGCTCCAGAATATCCT
CATTGCGATATGCCGCAGGGAGGAGTGACTTGGGATGGTG
GGTCCACTAATGATCTGCCCGATG

and it generated light with a rate enhancement of 169.3-fold relative to the reaction in the absence of deoxyribozyme. The blocking oligo used was REV2 (CATCGGGCAGATCATTAGTG, SEQ ID NO. 10).

The sixth most abundant deoxyribozyme was

(SEQ ID NO. 20)
GGAAGAAATGTAGAGGAAACAGTGACTCTGCAGAATATCC
TCACTGCGTAGTGGGGCAGGGGGGAGTGACTTGGGATGGG
GGCTACACTAATGATCTGCCCGATG

and it generated light with a rate enhancement of 186.5-fold relative to the reaction in the absence of deoxyribozyme. The blocking oligo used was REV2 (CATCGGGCAGATCATTAGTG, SEQ ID NO. 10).

The seventh most abundant deoxyribozyme was

(SEQ ID NO. 21)
GGAAGAAACGCGACGACACAGTAATGATGCGGAATATCCC
CCCTTCATAGGGCGAAAGGGGGGAGTGACTTGGGATGGGG
GTTACACTAATGATCTGCCCGATG

and it generated light with a rate enhancement of 116.7-fold relative to the reaction in the absence of deoxyribozyme. The blocking oligo used was REV2 (CATCGGGCAGATCATTAGTG, SEQ ID NO. 10).

The eighth most abundant deoxyribozyme was

(SEQ ID NO. 22)
GGCAGAGATGGCAACGTCAACATGAGGATGCCGCATATCC
CCAGTGCACACTTGGGCAGTGGGGAGTGACTTGGGATGGG
GGAAACACTAATGATCTGCCCGATG

and it generated light with a rate enhancement of 155.8-fold relative to the reaction in the absence of deoxyribozyme. The blocking oligo used was REV2 (CATCGGGCAGATCATTAGTG, SEQ ID NO. 10).

The ninth most abundant deoxyribozyme was

(SEQ ID NO. 23)
GGAAGATATGGCGGTGACTCTTCGAAGCTGCCGAATATCC
CCCGTGCGCGGAGCCTCAGGGGGGAGTGACTTGGGATGGG
GGGTCCACTAATGATCTGCCCGATG

and it generated light with a rate enhancement of 135.5-fold relative to the reaction in the absence of deoxyribozyme. The blocking oligo used was REV2 (CATCGGGCAGATCATTAGTG, SEQ ID NO. 10).

The tenth most abundant deoxyribozyme was

(SEQ ID NO. 24)
GGAAGAAATGCCGACCTCCCAGGGTGGAGGCTGAATATCCACCATGGTC
CGGAACGGATGGGGGAGTGACTTGGGATGGGGGAACCACTAATGATCTG
CCCGATG

and it generated light with a rate enhancement of 152.6-fold relative to the reaction in the absence of deoxyribozyme. The blocking oligo used was REV2 (CATCGGGCAGATCATTAGTG, SEQ ID NO. 10).

### Example 7: Identification of the Catalytic Core of the Deoxyribozyme

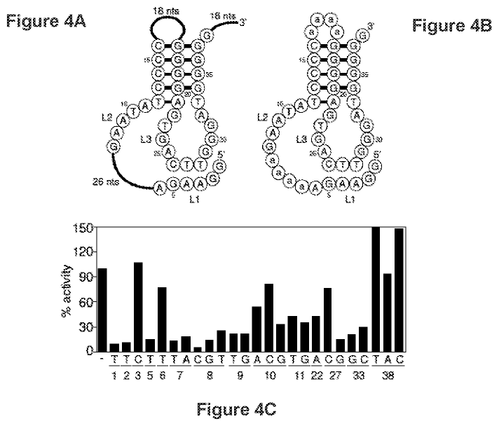

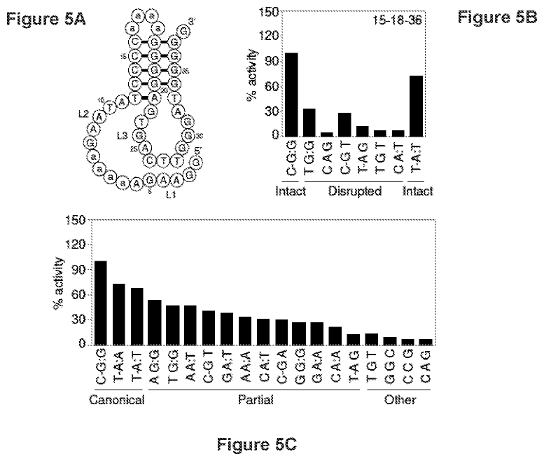

To identify the catalytic core of the deoxyribozyme, variable regions in the sequence alignment of deoxyribozymes from the reselection (Example 1) were either deleted or replaced with AAAA linkers. The minimized deoxyribozyme also contained a T to C mutation at position 15 in helix 1 (numbering corresponds to that used in FIG. 4) which occurred frequently in deoxyribozymes from the reselection. This deoxyribozyme was tested as described in Examples 3 and 4, except using 0.25 mM rather than 1 mM CDP-Star™ substrate.

The sequence of the minimized catalytic core of the deoxyribozyme is

(SEQ ID NO. 25)
GGAAGAAAAAGAATATCCCCAAAAGGGGAGTGACTTGGGATGGGGG

The amount of product in the ligation assay was 34.6% relative to a control oligonucleotide containing a 5′ phosphate. It also generated 128-fold more light than the background reaction in the absence of deoxyribozyme in the light-production assay. The catalytic activities of the variants described in Examples 8-13 are expressed relative to the activity of this construct.

### Example 8: Confirming the Secondary Structure Model of the Deoxyribozyme

To confirm the triple helix in the secondary structure model of the deoxyribozyme, a series of mutants were constructed in which a proposed base triple involving positions 15, 18, and 36 (numbering corresponds to that used in FIG. 4) was disrupted in various ways in single and double mutants, and restored in a triple mutant made up of mutations that by themselves disrupt the triple. Deoxyribozymes were tested as described in Example 4, except using 0.25 mM rather than 1 mM CDP-Star™ substrate.

The first mutant in the series

(SEQ ID NO. 25)
GGAAGAAAAAGAATATCCCCAAAAGGGGAGTGACTTGGGATGGGGG

contains a canonical triple (15C 18G 36G), and it generated 100% as much light as the most common minimized catalytic core in the reselection (SEQ ID NO. 25, which is the same sequence).

The second mutant in the series

(SEQ ID NO. 26)
GGAAGAAAAAGAATATCCTCAAAAGGGGAGTGACTTGGGATGGGGG

contains a disrupted triple (15T 18G 36G), and it generated 33.6% as much light as the most common minimized catalytic core in the reselection (SEQ ID NO. 25).

The third mutant in the series

(SEQ ID NO. 27)
GGAAGAAAAAGAATATCCCCAAAAGAGGAGTGACTTGGGATGGGGG

contains a disrupted triple (15C 18A 36G), and it generated 4.9% as much light as the most common minimized catalytic core in the reselection (SEQ ID NO. 25).

The fourth mutant in the series

(SEQ ID NO. 28)
GGAAGAAAAAGAATATCCCCAAAAGGGGAGTGACTTGGGATGGTGG

contains a disrupted triple (15C 18G 36T), and it generated 28.3% as much light as the most common minimized catalytic core in the reselection (SEQ ID NO. 25).

The fifth mutant in the series

(SEQ ID NO. 29)
GGAAGAAAAAGAATATCCTCAAAAGAGGAGTGACTTGGGATGGGGG

contains a disrupted triple (15T 18A 36G), and it generated 11.8% as much light as the most common minimized catalytic core in the reselection (SEQ ID NO. 25).

The sixth mutant in the series

SEQ ID NO: 20 =
(SEQ ID NO. 30)
GGAAGAAAAAGAATATCCTCAAAAGGGGAGTGACTTGGGATGGTGG

contains a disrupted triple (15T 18G 36T), and it generated 7.1% as much light as the most common minimized catalytic core in the reselection (SEQ ID NO. 25).

The seventh mutant in the series

(SEQ ID NO. 31)
GGAAGAAAAAGAATATCCCCAAAAGAGGAGTGACTTGGGATGGTGG

contains a disrupted triple (15C 18A 36T), and it generated 7.7% as much light as the most common minimized catalytic core in the reselection (SEC) ID NO. 25).

The eighth mutant in the series

(SEQ ID NO. 32)
GGAAGAAAAAGAATATCCTCAAAAGAGGAGTGACTTGGGATGGTGG

contains a canonical triple (15T 18A 36T), and it generated 72.9% as much light as the most common minimized catalytic core in the reselection (SEQ ID NO 25).

### Example 9: Catalytic Activity of Minimized Catalytic Cores Containing Point Mutations in Unpaired Regions which Occurred Frequently in Deoxyribozymes from the Reselection

Minimized catalytic cores containing point mutations in unpaired regions (positions 1-11, 22-33, and 38; numbering as in FIG. 4) which occurred frequently in deoxyribozymes from the reselection were tested as described in Example 4, except using 0.25 mM rather than 1 mM CDP-Star™ substrate. These catalytic cores correspond to sequences in which R¹ was replaced by AAAA, R² was replaced by AAAA, and R³ was deleted. The results are summarized in the text as well as in Table 2 below.

TABLE 2
Preferred nucleotides at unpaired positions in the deoxyribozyme.
Enrichment is the frequency of the mutation in the evolved library
divided by its frequency in the starting library. Only mutations with
enrichment values ≥3.1 × 10⁻³ are shown. Percent
activity is the amount of light produced by a variant of SEQ
ID NO. 25 containing the indicated mutation relative to SEQ
ID NO. 25 (which is normalized to a value of 100%).

| Position | Nucleotide | Enrichment | % activity |
|---|---|---|---|
| 1 | G | 1.2 | 100 |
| | T | 4.7 × 10⁻³ | 9.9 |
| 2 | G | 1.2 | 100 |
| | T | 3.1 × 10⁻³ | 11 |
| 3 | A | 1.2 | 100 |
| | C | 1.1 | 106.2 |
| 4 | A | 1.3 | 100 |
| 5 | G | 1.3 | 100 |
| | T | 3.6 × 10⁻³ | 14 |
| 6 | A | 1.3 | 100 |
| | T | 3 × 10⁻¹ | 77 |
| 7 | G | 1.7 | 100 |
| | T | 2.7 × 10⁻² | 13.1 |
| | A | 3.3 × 10⁻³ | 18.5 |
| 8 | A | 1.3 | 100 |
| | C | 1 | 5.3 |
| | G | 1.4 × 10⁻¹ | 13.2 |
| | T | 1.1 × 10⁻¹ | 25 |
| 9 | A | 1.5 | 100 |
| | T | 1.4 × 10⁻¹ | 21.4 |
| | G | 1.8 × 10⁻² | 21.9 |
| 10 | T | 1.4 | 100 |
| | A | 1.4 × 10⁻¹ | 54.7 |
| | C | 5.6 × 10⁻² | 81.5 |
| | G | 1.7 × 10⁻² | 32.7 |
| 11 | A | 1.4 | 100 |
| | T | 5 × 10⁻¹ | 42.8 |
| | G | 1.6 × 10⁻¹ | 34.3 |
| 22 | G | 1.7 | 100 |
| | A | 8.5 × 10⁻³ | 43 |
| 23 | T | 1.5 | 100 |
| 24 | G | 1.7 | 100 |
| 25 | A | 1.5 | 100 |
| 26 | C | 1.5 | 100 |
| 27 | T | 1.5 | 100 |
| | C | 1.3 × 10⁻² | 76 |
| | G | 4.7 × 10⁻³ | 14.1 |
| 28 | T | 1.5 | 100 |
| 29 | G | 1.7 | 100 |
| 30 | G | 1.7 | 100 |
| 31 | G | 1.8 | 100 |
| 32 | A | 1.5 | 100 |
| 33 | T | 1.5 | 100 |
| | G | 6.3 × 10⁻³ | 20.5 |
| | C | 5.9 × 10⁻³ | 29.7 |
| 38 | G | 1.6 | 100 |
| | T | 3.3 × 10⁻¹ | 156.4 |
| | A | 1.4 × 10⁻¹ | 94.1 |
| | C | 5.3 × 10⁻² | 148.2 |

The first construct in this series contains the 1T mutation. It has the sequence

(SEQ ID NO. 33)
TGAAGAAAAAGAATATCCCCAAAAGGGGAGTGACTTGGGATGGGGG

and it generated 9.9% as much light as the most common minimized catalytic core in the reselection (SEQ ID NO. 25).

The second construct in this series contains the 2T mutation. It has the sequence

(SEQ ID NO. 34)
GTAAGAAAAAGAATATCCCCAAAAGGGGAGTGACTTGGGATGGGGG

and it generated 11% as much light as the most common minimized catalytic core in the reselection (SEQ ID NO. 25).

The third construct in this series contains the 3C mutation. It has the sequence

(SEQ ID NO. 35)
GGCAGAAAAAGAATATCCCCAAAAGGGGAGTGACTTGGGATGGGGG

and it generated 106.2% as much light as the most common minimized catalytic core in the reselection (SEQ ID NO. 25).

The fourth construct in this series contains the 5T mutation. It has the sequence

(SEQ ID NO. 36)
GGAATAAAAAGAATATCCCCAAAAGGGGAGTGACTTGGGATGGGGG

and it generated 14% as much light as the most common minimized catalytic core in the reselection (SEQ ID NO. 25).

The fifth construct in this series contains the 6T mutation. It has the sequence

(SEQ ID NO. 37)
GGAAGTAAAAGAATATCCCCAAAAGGGGAGTGACTTGGGATGGGGG

and it generated 77% as much light as the most common minimized catalytic core in the reselection (SEQ ID NO. 25).

The sixth construct in this series contains the 7T mutation. It has the sequence

(SEQ ID NO. 38)
GGAAGAAAAATAATATCCCCAAAAGGGGAGTGACTTGGGATGGGGG

and it generated 13.1% as much light as the most common minimized catalytic core in the reselection (SEQ ID NO. 25).

The seventh construct in this series contains the 7A mutation. It has the sequence

(SEQ ID NO. 39)
GGAAGAAAAAAAATATCCCCAAAAGGGGAGTGACTTGGGATGGGGG

and it generated 18.5% as much light as the most common minimized catalytic core in the reselection (SEQ ID NO. 25).

The eighth construct in this series contains the 8C mutation. It has the sequence

(SEQ ID NO. 40)
GGAAGAAAAAGCATATCCCCAAAAGGGGAGTGACTTGGGATGGGGG

and it generated 5.3% as much light as the most common minimized catalytic core in the reselection (SEQ ID NO. 25).

The ninth construct in this series contains the 8G mutation. It has the sequence

(SEQ ID NO. 41)
GGAAGAAAAAGGATATCCCCAAAAGGGGAGTGACTTGGGATGGGGG

and it generated 13.2% as much light as the most common minimized catalytic core in the reselection (SEQ ID NO. 25).

The tenth construct in this series contains the 8T mutation. It has the sequence

(SEQ ID NO. 42)
GGAAGAAAAAGTATATCCCCAAAAGGGGAGTGACTTGGGATGGGGG

and it generated 25% as much light as the most common minimized catalytic core in the reselection (SEQ ID NO. 25).

The eleventh construct in this series contains the 9T mutation. It has the sequence

(SEQ ID NO. 43)
GGAAGAAAAAGATTATCCCCAAAAGGGGAGTGACTTGGGATGGGGG

and it generated 21.4% as much light as the most common minimized catalytic core in the reselection (SEQ ID NO. 25).

The twelfth construct in this series contains the 9G mutation. It has the sequence

(SEQ ID NO. 44)
GGAAGAAAAAGAGTATCCCCAAAAGGGGAGTGACTTGGGATGGGGG

and it generated 21.9% as much light as the most common minimized catalytic core in the reselection (SEQ ID NO. 25).

The thirteenth construct in this series contains the 10A mutation. It has the sequence

(SEQ ID NO. 45)
GGAAGAAAAAGAAAATCCCCAAAAGGGGAGTGACTTGGGATGGGGG

and it generated 54.7% as much light as the most common minimized catalytic core in the reselection (SEQ ID NO. 25).

The fourteenth construct in this series contains the 10C mutation. It has the sequence

(SEQ ID NO. 46)
GGAAGAAAAAGAACATCCCCAAAAGGGGAGTGACTTGGGATGGGGG

and it generated 81.5% as much light as the most common minimized catalytic core in the reselection (SEQ ID NO. 25).

The fifteenth construct in this series contains the 10G mutation. It has the sequence

(SEQ ID NO. 47)
GGAAGAAAAAGAAGATCCCCAAAAGGGGAGTGACTTGGGATGGGGG

and it generated 32.7% as much light as the most common minimized catalytic core in the reselection (SEQ ID NO. 25).

The sixteenth construct in this series contains the 11T mutation. It has the sequence

(SEQ ID NO. 48)
GGAAGAAAAAGAATTTCCCCAAAAGGGGAGTGACTTGGGATGGGGG

and it generated 42.8% as much light as the most common minimized catalytic core in the reselection (SEQ ID NO. 25).

The seventeenth construct in this series contains the 11G mutation. It has the sequence

(SEQ ID NO. 49)
GGAAGAAAAAGAATGTCCCCAAAAGGGGAGTGACTTGGGATGGGGG

and it generated 34.3% as much light as the most common minimized catalytic core in the reselection (SEQ ID NO. 25).

The eighteenth construct in this series contains the 22A mutation. It has the sequence

(SEQ ID NO. 50)
GGAAGAAAAAGAATATCCCCAAAAGGGGAATGACTTGGGATGGGGG

and it generated 43% as much light as the most common minimized catalytic core in the reselection (SEQ ID NO. 25).

The nineteenth construct in this series contains the 27C mutation. It has the sequence

(SEQ ID NO. 51)
GGAAGAAAAAGAATATCCCCAAAAGGGGAGTGACCTGGGATGGGGG

and it generated 76% as much light as the most common minimized catalytic core in the reselection (SEQ ID NO. 25).

The twentieth construct in this series contains the 27G mutation. It has the sequence

(SEQ ID NO. 52)
GGAAGAAAAAGAATATCCCCAAAAGGGGAGTGACGTGGGATGGGGG

and it generated 14.1% as much light as the most common minimized catalytic core in the reselection (SEQ ID NO. 25).

The twenty first construct in this series contains the 33G mutation. It has the sequence

(SEQ ID NO. 53)
GGAAGAAAAAGAATATCCCCAAAAGGGGAGTGACTTGGGAGGGGGG

and it generated 20.5% as much light as the most common minimized catalytic core in the reselection (SEQ ID NO. 25).

The twenty second construct in this series contains the 33C mutation. It has the sequence

(SEQ ID NO. 54)
GGAAGAAAAAGAATATCCCCAAAAGGGGAGTGACTTGGGACGGGGG

and it generated 29.7% as much light as the most common minimized catalytic core in the reselection (SEQ ID NO. 25).

The twenty third construct in this series contains the 38T mutation. It has the sequence

(SEQ ID NO. 55)
GGAAGAAAAAGAATATCCCCAAAAGGGGAGTGACTTGGGATGGGGT

and it generated 156.4% as much light as the most common minimized catalytic core in the reselection (SEQ ID NO. 25).

The twenty fourth construct in this series contains the 38A mutation. It has the sequence

(SEQ ID NO. 56)
GGAAGAAAAAGAATATCCCCAAAAGGGGAGTGACTTGGGATGGGGA

and it generated 94.1% as much light as the most common minimized catalytic core in the reselection (SEQ ID NO. 25).

The twenty fifth construct in this series contains the 38C mutation. It has the sequence

(SEQ ID NO. 57)
GGAAGAAAAAGAATATCCCCAAAAGGGGAGTGACTTGGGATGGGGC

and it generated 148.2% as much light as the most common minimized catalytic core in the reselection (SEQ ID NO. 25).

### Example 10: Catalytic Activity of Minimized Catalytic Cores Containing Different Combinations of Canonical Base Pairs and Base Triples

Minimized catalytic cores containing different combinations of canonical base pairs (at the 12-21 base pair) and base triples (at the 13-20-34, 14-19-35, 15-18-36, and 16-17-37 base triples; numbering as in FIG. 4) were tested as described in Example 4, except using 0.25 mM rather than 1 mM CDP-Star™ substrate. These catalytic cores correspond to sequences in which R¹ was replaced by AAAA, R² was replaced by AAAA, and R³ was deleted. The results are summarized in the text as well as in Table 3 below.

TABLE 3
Activity of deoxyribozymes containing helices with different sequences
and lengths. Percent activity is the amount of light produced by
a variant of SEQ ID: 15 containing the indicated mutations relative
to SEQ ID: 15 (which is normalized to a value of 100%).

| Positions | Pair or triple | % activity |
|---|---|---|
| 12-21 | T-A | 100 |
| | A-T | 96 |
| | C-G | 12.2 |
| | G-C | 51.6 |
| 13-20-34 | All C-G:G | 100 |
| 14-19-35 | | |
| 15-18-36 | | |
| 16-17-37 | | |
| 13-20-34 | T-A:A | 15.1 |
| | T-A:T | 11 |
| 14-19-35 | T-A:A | 119.1 |
| | T-A:T | 45.4 |
| 15-18-36 | T-A:A | 61 |
| | T-A:T | 72.9 |
| 16-17-37 | T-A:A | 98.6 |
| | T-A:T | 141.7 |
| 14-19-35 | All T-A:A | 27.4 |
| 15-18-36 | | |
| 16-17-37 | | |
| 14-19-35 | All T-A:T | 34.9 |
| 15-18-36 | | |
| 16-17-37 | | |
| 13-20-34 | All T-A:A | 14.1 |
| 14-19-35 | | |
| 15-18-36 | | |
| 16-17-37 | | |
| 13-20-34 | All T-A:T | 12.5 |
| 14-19-35 | | |
| 15-18-36 | | |
| 16-17-37 | | |
| 16-17-37 | Deleted | 24.3 |
| 15-18-36 | Deleted | 3.3 |
| 16-17-37 | | |
| 14-19-35 | Deleted | 3.1 |
| 15-18-36 | | |
| 16-17-37 | | |

The first construct in this series contains T at position 12 and A at position 21. It has the sequence

(SEQ ID NO. 25)
GGAAGAAAAAGAATATCCCCAAAAGGGGAGTGACTTGGGATGGGGG

and it generated 100% as much light as the most common minimized catalytic core in the reselection (SEQ ID NO. 25, which is the same sequence).

The second construct in this series contains A at position 12 and T at position 21. It has the sequence

(SEQ ID NO. 58)
GGAAGAAAAAGAATAACCCCAAAAGGGGTGTGACTTGGGATGGGGG

and it generated 96% as much light as the most common minimized catalytic core in the reselection (SEQ ID NO. 25).

The third construct in this series contains C at position 12 and G at position 21. It has the sequence

(SEQ ID NO. 59)
GGAAGAAAAAGAATACCCCCAAAAGGGGGGTGACTTGGGATGGGGG

and it generated 12.2% as much light as the most common minimized catalytic core in the reselection (SEQ ID NO. 25).

The fourth construct in this series contains G at position 12 and C at position 21. It has the sequence

(SEQ ID NO. 60)
GGAAGAAAAAGAATAGCCCCAAAAGGGGCGTGACTTGGGATGGGGG

and it generated 51.6% as much light as the most common minimized catalytic core in the reselection (SEQ ID NO. 25).

The fifth construct in this series contains C-C:G base triples at positions 13-20-34, 14-19-35, 15-18-36, and 16-17-37. It has the sequence

(SEQ ID NO. 25)
GGAAGAAAAAGAATATCCCCAAAAGGGGAGTGACTTGGGATGGGGG

and it generated 100% as much light as the most common minimized catalytic core in the reselection (SEQ ID NO. 25, which is the same sequence).

The sixth construct in this series contains a T-A:A base triple at position 13-20-34 and C-G:G triples at the other positions in the triple helix. It has the sequence

(SEQ ID NO. 61)
GGAAGAAAAAGAATATTCCCAAAAGGGAAGTGACTTGGGATAGGGG

and it generated 15.1% as much light as the most common minimized catalytic core in the reselection (SEQ ID NO. 25).

The seventh construct in this series contains a T-A:T base triple at position 13-20-34 and C-G:G triples at the other positions in the triple helix. It has the sequence

(SEQ ID NO. 62)
GGAAGAAAAAGAATATTCCCAAAAGGGAAGTGACTTGGGATTGGGG

and it generated 11% as much light as the most common minimized catalytic core in the reselection (SEQ ID NO. 25).

The eighth construct in this series contains a T-A:A base triple at position 14-19-35 and C-G:G triples at the other positions in the triple helix. It has the sequence

(SEQ ID NO. 63)
GGAAGAAAAAGAATATCTCCAAAAGGAGAGTGACTTGGGATGAGGG

and it generated 119.1% as much light as the most common minimized catalytic core in the reselection (SEQ ID NO. 25).

The ninth construct in this series contains a T-A:T base triple at position 14-19-35 and C-G:G triples at the other positions in the triple helix. It has the sequence

(SEQ ID NO. 64)
GGAAGAAAAAGAATATCTCCAAAAGGAGAGTGACTTGGGATGTGGG

and it generated 45.4% as much light as the most common minimized catalytic core in the reselection (SEQ ID NO. 25).

The tenth construct in this series contains a T-A:A base triple at position 15-18-36 and C-G:G triples at the other positions in the triple helix. It has the sequence

(SEQ ID NO. 65)
GGAAGAAAAAGAATATCCTCAAAAGAGGAGTGACTTGGGATGGAGG

and it generated 61% as much light as the most common minimized catalytic core in the reselection (SEQ ID NO. 25).

The eleventh construct in this series contains a T-A:T base triple at position 15-18-36 and C-G:G triples at the other positions in the triple helix. It has the sequence

(SEQ ID NO. 66)
GGAAGAAAAAGAATATCCTCAAAAGAGGAGTGACTTGGGATGGTGG

and it generated 72.9% as much light as the most common minimized catalytic core in the reselection (SEQ ID NO. 25).

The twelfth construct in this series contains a T-A:A base triple at position 16-17-37 and C-G:G triples at the other positions in the triple helix. It has the sequence

(SEQ ID NO. 67)
GGAAGAAAAAGAATATCCCTAAAAAGGGAGTGACTTGGGATGGGAG

and it generated 98.6% as much light as the most common minimized catalytic core in the reselection (SEQ ID NO. 25).

The thirteenth construct in this series contains a T-A:T base triple at position 16-17-37 and C-G:G triples at the other positions in the triple helix. It has the sequence

(SEQ ID NO. 68)
GGAAGAAAAAGAATATCCCTAAAAAGGGAGTGACTTGGGATGGGTG

and it generated 141.7% as much light as the most common minimized catalytic core in the reselection (SEQ ID NO. 25).

The fourteenth construct in this series contains a C-G:G base triple at position 13-20-34 and T-A:A base triples at position 14-19-35, 15-18-36, and 16-17-37. It has the sequence

(SEQ ID NO. 69)
GGAAGAAAAAGAATATCTTTAAAAAAAGAGTGACTTGGGATGAAAG

and it generated 27.4% as much light as the most common minimized catalytic core in the reselection (SEQ ID NO. 25).

The fifteenth construct in this series contains a C-G:G base triple at position 13-20-34 and T-A:T base triples at position 14-19-35, 15-18-36, and 16-17-37. It has the sequence

(SEQ ID NO. 70)
GGAAGAAAAAGAATATCTTTAAAAAAAGAGTGACTTGGGATGTTTG

and it generated 34.9% as much light as the most common minimized catalytic core in the reselection (SEQ ID NO. 25).

The sixteenth construct in this series contains T-A:A base triples at all positions in the triple helix. It has the sequence

(SEQ ID NO. 71)
GGAAGAAAAAGAATATTTTTAAAAAAAAAGTGACTTGGGATAAAAG

and it generated 14.1% as much light as the most common minimized catalytic core in the reselection (SEQ ID NO. 25).

The seventeenth construct in this series contains T-A:T base triples at all positions in the triple helix. It has the sequence

(SEQ ID NO. 72)
GGAAGAAAAAGAATATTTTTAAAAAAAAAGTGACTTGGGATTTTTG

and it generated 12.5% as much light as the most common minimized catalytic core in the reselection (SEQ ID NO. 25).

The eighteenth construct in this series contains a deleted triple at position 16-17-37 and C-G:G triples at the other positions in the triple helix. It has the sequence

(SEQ ID NO. 73)
GGAAGAAAAAGAATATCCCAAAAGGGAGTGACTTGGGATGGGG

and it generated 24.3% as much light as the most common minimized catalytic core in the reselection (SEQ ID NO. 25).

The nineteenth construct in this series contains deleted triples at positions 16-17-37 and 15-18-36, and C-G:G triples at the other positions in the triple helix. It has the sequence

(SEQ ID NO. 74)
GGAAGAAAAAGAATATCCAAAAGGAGTGACTTGGGATGGG

and it generated 3.3% as much light as the most common minimized catalytic core in the reselection (SEQ ID NO. 25).

The twentieth construct in this series contains deleted triples at positions 16-17-37, 15-18-36, and 14-19-35, and a C:G:G triple at position 13-20-34 in the triple helix. it has the sequence

(SEQ ID NO. 75)
GGAAGAAAAAGAATATCAAAAGAGTGACTTGGGATGG

and it generated 3.1% as much light as the most common minimized catalytic core in the reselection (SEQ ID NO. 25).

### Example 11: Catalytic Activity of Minimized Catalytic Cores Containing Partially or Completely Disrupted Base Triples

Minimized catalytic cores containing different partially or completely disrupted triples at the 13-20-34, 14-19-35, 15-18-36, and 16-17-37 base triples (numbering as in FIG. 4) were tested as described in Example 4, except using 0.25 mM rather than 1 mM CDP-Star™ substrate. These catalytic cores correspond to sequences in which R¹ was replaced by AAAA, R² was replaced by AAAA, and R³ was deleted. The results are summarized in the text as well as in Table 4 below.

TABLE 4
Activity of deoxyribozymes containing disrupted base triples.
Percent activity is the amount of light produced by a variant
of SEQ ID NO. 25 containing the indicated mutations relative
to SEQ ID NO. 25 (which is normalized to a value of 100%). Asterisks
indicate base triples with enrichment values (frequency in evolved library
divided by frequency in starting library) ≥10⁻³.

| Sequence | Type of | % activity | | | |
|---|---|---|---|---|---|
| of triple | triple | 13-20-34 | 14-19-35 | 15-18-36 | 16-17-37 |
| C-G:G | Canonical | 100* | 100* | 100* | 100* |
| T-A:T | | 11* | 45.4 | 72.9* | 141.7* |
| T-A:A | | 15.1 | 119.1* | 61* | 98.6* |
| A G:G | Partial | 64* | 36* | 22.4* | 92.3 |
| G G:G | Hoogsteen | 25 | 12.2 | 36 | 32.1 |
| T G:G | | 48.6 | 32 | 33.6* | 73.1* |
| A A:A | | 19.5 | 26.8 | 25.9 | 59.3 |
| C A:A | | 14.9 | 16.5 | 13.6 | 41.2 |
| G A:A | | 17.7 | 50.6 | 7.9* | 26.9 |
| A A:T | | 28.2 | 19.5 | 33.1 | 104.3 |
| C A:T | | 2.8 | 2.9 | 7.7* | 107.4 |
| G A:T | | 15 | 24.4 | 29 | 82.7 |
| C-G A | Partial | 6.8 | 44.1* | 18.4 | 50.9 |
| C-G T | Watson- | 6.4 | 54.2 | 28.3 | 71 |
| T-A G | Crick | 3.8 | 3.5 | 11.8 | 29.6 |
| A-T A | Other | — | — | — | 89.1 |
| G-C A | | — | — | — | 38.7 |
| T G T | | 3.6 | 3.7 | 7.1 | 35.2 |
| G T A | | — | — | — | 22.7 |
| G G C | | 6.9 | 6.8 | 9.4* | 13.5 |
| C C G | | 6.8 | 6.5 | 5.8 | 7.2 |
| C A G | | 3.1 | 2.9 | 4.9 | 11.3 |

The first construct in the series contains an A G:G triple at position 13-20-34 in the triple helix. It has the sequence

(SEQ ID NO. 76)
GGAAGAAAAAGAATATACCCAAAAGGGGAGTGACTTGGGATGGGGG

and it generated 64% as much light as the most common minimized catalytic core in the reselection (SEQ ID NO. 25).

The next construct in the series contains a G G:G triple at position 13-20-34 in the triple helix. It has the sequence

(SEQ ID NO. 77)
GGAAGAAAAAGAATATGCCCAAAAGGGGAGTGACTTGGGATGGGGG

and it generated 25% as much light as the most common minimized catalytic core in the reselection (SEQ ID NO. 25).

The next construct in the series contains a T G:G triple at position 13-20-34 in the triple helix. It has the sequence

(SEQ ID NO. 78)
GGAAGAAAAAGAATATTCCCAAAAGGGGAGTGACTTGGGATGGGGG

and it generated 48.6% as much light as the most common minimized catalytic core in the reselection (SEQ ID NO. 25).

The next construct in the series contains a A A:A triple at position 13-20-34 in the triple helix. It has the sequence

(SEQ ID NO. 79)
GGAAGAAAAAGAATATACCCAAAAGGGAAGTGACTTGGGATAGGGG

and it generated 19.5% as much light as the most common minimized catalytic core in the reselection (SEQ ID NO. 25).

The next construct in the series contains a C A:A triple at position 13-20-34 in the triple helix. It has the sequence

(SEQ ID NO. 80)
GGAAGAAAAAGAATATCCCCAAAAGGGAAGTGACTTGGGATAGGGG

and it generated 14.9% as much light as the most common minimized catalytic core in the reselection (SEQ ID NO. 25).

The next construct in the series contains a G A:A triple at position 13-20-34 in the triple helix. It has the sequence

(SEQ ID NO. 81)
GGAAGAAAAAGAATATGCCCAAAAGGGAAGTGACTTGGGATAGGGG

and it generated 17.7% as much light as the most common minimized catalytic core in the reselection (SEQ ID NO. 25).

The next construct in the series contains a A A:T triple at position 13-20-34 in the triple helix. It has the sequence

(SEQ ID NO. 82)
GGAAGAAAAAGAATATACCCAAAAGGGAAGTGACTTGGGATTGGGG

and it generated 28.2% as much light as the most common minimized catalytic core in the reselection (SEQ ID NO. 25).

The next construct in the series contains a C A:T triple at position 13-20-34 in the triple helix. It has the sequence

(SEQ ID NO. 83)
GGAAGAAAAAGAATATCCCCAAAAGGGAAGTGACTTGGGATTGGGG

and it generated 2.8% as much light as the most common minimized catalytic core in the reselection (SEQ ID NO. 25).

The next construct in the series contains a G A:T triple at position 13-20-34 in the triple helix. It has the sequence

(SEQ ID NO. 84)
GGAAGAAAAAGAATATGCCCAAAAGGGAAGTGACTTGGGATTGGGG

and it generated 15% as much light as the most common minimized catalytic core in the reselection (SEQ ID NO. 25).

The next construct in the series contains a C-G A triple at position 13-20-34 in the triple helix. It has the sequence

(SEQ ID NO. 85)
GGAAGAAAAAGAATATCCCCAAAAGGGGAGTGACTTGGGATAGGGG

and it generated 6.8% as much light as the most common minimized catalytic core in the reselection (SEQ ID NO. 25).

The next construct in the series contains a C-G T triple at position 13-20-34 in the triple helix. It has the sequence

(SEQ ID NO. 86)
GGAAGAAAAAGAATATCCCCAAAAGGGGAGTGACTTGGGATTGGGG

and it generated 6.4% as much light as the most common minimized catalytic core in the reselection (SEQ ID NO. 25).

The next construct in the series contains a T-A G triple at position 13-20-34 in the triple helix. It has the sequence

(SEQ ID NO. 87)
GGAAGAAAAAGAATATTCCCAAAAGGGAAGTGACTTGGGATGGGGG

and it generated 3.8% as much light as the most common minimized catalytic core in the reselection (SEQ ID NO. 25).

The next construct in the series contains a T G T triple at position 13-20-34 in the triple. helix. It has the sequence

(SEQ ID NO. 88)
GGAAGAAAAAGAATATTCCCAAAAGGGGAGTGACTTGGGATTGGGG

and it generated 3.6% as much light as the most common minimized catalytic core in the reselection (SEQ ID NO. 25).

The next construct in the series contains a G G C triple at position 13-20-34 in the triple helix. It has the sequence

(SEQ ID NO. 89)
GGAAGAAAAAGAATATGCCCAAAAGGGGAGTGACTTGGGATCGGGG

and it generated 6.9% as much light as the most common minimized catalytic core in the reselection (SEQ ID NO. 25).

The next construct in the series contains a C C G triple at position 13-20-34 in the triple helix. It has the sequence

(SEQ ID NO. 90)
GGAAGAAAAAGAATATCCCCAAAAGGGCAGTGACTTGGGATGGGGG

and it generated 6.8% as much light as the most common minimized catalytic core in the reselection (SEQ ID NO. 25).

The next construct in the series contains a C A G triple at position 13-20-34 in the triple helix. It has the sequence

(SEQ ID NO. 91)
GGAAGAAAAAGAATATCCCCAAAAGGGAAGTGACTTGGGATGGGGG

and it generated 3.1% as much light as the most common minimized catalytic core in the reselection (SEQ ID NO. 25).

The next construct in the series contains a A G:G triple at position 14-19-35 in the triple helix. It has the sequence

(SEQ ID NO. 92)
GGAAGAAAAAGAATATCACCAAAAGGGGAGTGACTTGGGATGGGGG

and it generated 36% as much light as the most common minimized catalytic core in the reselection (SEQ ID NO. 25).

The next construct in the series contains a G G:G triple at position 14-19-35 in the triple helix. It has the sequence

(SEQ ID NO. 93)
GGAAGAAAAAGAATATCGCCAAAAGGGGAGTGACTTGGGATGGGGG

and it generated 12.2% as much light as the most common minimized catalytic core in the reselection (SEQ ID NO. 25).

The next construct in the series contains a T G:G triple at position 14-19-35 in the triple helix. It has the sequence

(SEQ ID NO. 94)
GGAAGAAAAAGAATATCTCCAAAAGGGGAGTGACTTGGGATGGGGG

and it generated 49% as much light as the most common minimized catalytic core in the reselection (SEQ ID NO. 25).

The next construct in the series contains a A A:A triple at position 14-19-35 in the triple helix. It has the sequence

(SEQ ID NO. 95)
GGAAGAAAAAGAATATCACCAAAAGGAGAGTGACTTGGGATGAGGG

and it generated 26.8% as much light as the most common minimized catalytic core in the reselection (SEQ ID NO. 25).

The next construct in the series contains a C A:A triple at position 14-19-35 in the triple helix. It has the sequence

(SEQ ID NO. 96)
GGAAGAAAAAGAATATCCCCAAAAGGAGAGTGACTTGGGATGAGGG

and it generated 16.5% as much light as the most common minimized catalytic core in the reselection (SEQ ID NO. 25).

The next construct in the series contains a G A:A triple at position 14-19-35 in the triple helix. It has the sequence

(SEQ ID NO. 97)
GGAAGAAAAAGAATATCGCCAAAAGGAGAGTGACTTGGGATGAGGG

and it generated 50.6% as much light as the most common minimized catalytic core in the reselection (SEQ ID NO. 25).

The next construct in the series contains a A A:T triple at position 14-19-35 in the triple helix. It has the sequence

(SEQ ID NO. 98)
GGAAGAAAAAGAATATCACCAAAAGGAGAGTGACTTGGGATGTGGG

and it generated 19.5% as much light as the most common minimized catalytic core in the reselection (SEQ NO. 25).

The next construct in the series contains a C A:T triple at position 14-19-35 in the triple helix. It has the sequence

(SEQ ID NO. 99)
GGAAGAAAAAGAATATCCCCAAAAGGAGAGTGACTTGGGATGTGGG

and it generated 2.9% as much light as the most common minimized catalytic core in the reselection (SEQ ID NO. 25).

The next construct in the series contains a G A:T triple at position 14-19-35 in the triple helix. It has the sequence

(SEQ ID NO. 100)
GGAAGAAAAAGAATATCGCCAAAAGGAGAGTGACTTGGGATGTGGG

and it generated 24.4% as much light as the most common minimized catalytic core in the reselection (SEQ ID NO. 25).

The next construct in the series contains a C-G A triple at position 14-19-35 in the triple helix. It has the sequence

(SEQ ID NO. 101)
GGAAGAAAAAGAATATCCCCAAAAGGGGAGTGACTTGGGATGAGGG

and it generated 52.3% as much light as the most common minimized catalytic core in the reselection (SEQ ID NO. 25).

The next construct in the series contains a C-G T triple at position 14-19-35 in the triple helix. It has the sequence

(SEQ ID NO. 102)
GGAAGAAAAAGAATATCCCCAAAAGGGGAGTGACTTGGGATGTGGG

and it generated 54.2% as much light as the most common minimized catalytic core in the reselection (SEQ ID NO. 25).

The next construct in the series contains a T-A G triple at position 14-19-35 in the triple helix. It has the sequence

(SEQ ID NO. 103)
GGAAGAAAAAGAATATCTCCAAAAGGAGAGTGACTTGGGATGGGGG

and it generated 3.5% as much light as the most common minimized catalytic core in the reselection (SEQ ID NO. 25).

The next construct in the series contains a T G T triple at position 14-19-35 in the triple helix. It has the sequence

(SEQ ID NO. 104)
GGAAGAAAAAGAATATCTCCAAAAGGGGAGTGACTTGGGATGTGGG

and it generated 3.7% as much light as the most common minimized catalytic core in the reselection (SEQ ID NO. 25).

The next construct in the series contains a G G C triple at position 14-19-35 in the triple helix. It has the sequence

(SEQ ID NO. 105)
GGAAGAAAAAGAATATCGCCAAAAGGGGAGTGACTTGGGATGCGGG

and it generated 6.8% as much light as the most common minimized catalytic core in the reselection (SEQ ID NO. 25).

The next construct in the series contains a C C G triple at position 14-19-35 in the triple helix. It has the sequence

(SEQ ID NO. 106)
GGAAGAAAAAGAATATCCCCAAAAGGCGAGTGACTTGGGATGGGGG

and it generated 6.5% as much light as the most common minimized catalytic core in the reselection (SEQ ID NO. 25).

The next construct in the series contains a C A G triple at position 14-19-35 in the triple helix. It has the sequence

(SEQ ID NO. 107)
GGAAGAAAAAGAATATCCCCAAAAGGAGAGTGACTTGGGATGGGGG

and it generated 2.9% as much light as the most common minimized catalytic core in the reselection (SEQ ID NO. 25).

The next construct in the series contains a A G:G triple at position 15-18-36 in the triple helix. It has the sequence

(SEQ ID NO. 108)
GGAAGAAAAAGAATATCCACAAAAGGGGAGTGACTTGGGATGGGGG

and it generated 22.4% as much light as the most common minimized catalytic core in the reselection (SEQ ID NO. 25).

The next construct in the series contains a G G:G triple at position 15-18-36 in the triple helix. It has the sequence

(SEQ ID NO. 109)
GGAAGAAAAAGAATATCCGCAAAAGGGGAGTGACTTGGGATGGGGG

and it generated 36% as much light as the most common minimized catalytic core in the reselection (SEQ ID NO. 25).

The next construct in the series contains a T G:G triple at position 15-18-36 in the triple helix. It has the sequence

(SEQ ID NO. 26)
GGAAGAAAAAGAATATCCTCAAAAGGGGAGTGACTTGGGATGGGGG

and it generated 33.6% as much light as the most common minimized catalytic core in the reselection (SEQ ID NO. 25).

The next construct in the series contains a A A:A triple at position 15-18-36 in the triple helix. It has the sequence

(SEQ ID NO. 110)
GGAAGAAAAAGAATATCCACAAAAGAGGAGTGACTTGGGATGGAGG

and it generated 25.9% as much light as the most common minimized catalytic core in the reselection (SEQ ID NO. 25).

The next construct in the series contains a C A:A triple at position 15-18-36 in the triple helix. It has the sequence

(SEQ ID NO. 111)
GGAAGAAAAAGAATATCCCCAAAAGAGGAGTGACTTGGGATGGAGG

and it generated 13.6% as much light as the most common minimized catalytic core in the reselection (SEQ ID NO. 25).

The next construct in the series contains a G A:A triple at position 15-18-36 in the triple helix. It has the sequence

(SEQ ID NO. 112)
GGAAGAAAAAGAATATCCGCAAAAGAGGAGTGACTTGGGATGGAGG

and it generated 7.9% as much light as the most common minimized catalytic core in the reselection (SEQ ID NO. 25).

The next construct in the series contains a A A:T triple at position 15-18-36 in the triple helix. It has the sequence

(SEQ ID NO. 113)
GGAAGAAAAAGAATATCCACAAAAGAGGAGTGACTTGGGATGGTGG

and it generated 33.1% as much light as the most common minimized catalytic core in the reselection (SEQ ID NO. 25).

The next construct in the series contains a C A:T triple at position 15-18-36 in the triple helix. It has the sequence

(SEQ ID NO. 31)
GGAAGAAAAAGAATATCCCCAAAAGAGGAGTGACTTGGGATGGTGG

and it generated 7.7% as much light as the most common minimized catalytic core in the reselection (SEQ ID NO. 25).

The next construct in the series contains a G A:T triple at position 15-18-36 in the triple helix. It has the sequence

(SEQ ID NO. 114)
GGAAGAAAAAGAATATCCGCAAAAGAGGAGTGACTTGGGATGGTGG

and it generated 29% as much light as the most common minimized catalytic core in the reselection (SEQ ID NO. 25).

The next construct in the series contains a C-G A triple at position 15-18-36 in the triple helix. It has the sequence

(SEQ ID NO. 115)
GGAAGAAAAAGAATATCCCCAAAAGGGGAGTGACTTGGGATGGAGG

and it generated 18.4% as much light as the most common minimized catalytic core in the reselection (SEQ ID NO. 25).

The next construct in the series contains a C-G T triple at position 15-18-36 in the triple helix. It has the sequence

(SEQ ID NO. 28)
GGAAGAAAAAGAATATCCCCAAAAGGGGAGTGACTTGGGATGGTGG

and it generated 28.3% as much light as the most common minimized catalytic core in the reselection (SEQ ID NO. 25).

The next construct in the series contains a T-A G triple at position 15-18-36 in the triple helix. It has the sequence

(SEQ ID NO. 29)
GGAAGAAAAAGAATATCCTCAAAAGAGGAGTGACTTGGGATGGGGG

and it generated 11.8% as much light as the most common minimized catalytic core in the reselection (SEQ ID NO. 25).

The next construct in the series contains a T G T triple at position 15-18-36 in the triple helix. It has the sequence

(SEQ ID NO. 30)
GGAAGAAAAAGAATATCCTCAAAAGGGGAGTGACTTGGGATGGTGG

and it generated 7.1% as much light as the most common minimized catalytic core in the reselection (SEQ ID NO. 25).

The next construct in the series contains a G G C triple at position 15-18-36 in the triple helix. It has the sequence

(SEQ ID NO. 116)
GGAAGAAAAAGAATATCCGCAAAAGGGGAGTGACTTGGGATGGCGG

and it generated 9.4% as much light as the most common minimized catalytic core in the reselection (SEQ ID NO. 25).

The next construct in the series contains a C C G triple at position 15-18-36 in the triple helix. It has the sequence

(SEQ ID NO. 117)
GGAAGAAAAAGAATATCCCCAAAAGCGGAGTGACTTGGGATGGGGG

and it generated 58% as much light as the most common minimized catalytic core in the reselection (SEQ ID NO. 25).

The next construct in the series contains a C A G triple at position 15-18-36 in the triple helix. It has the sequence

(SEQ ID NO. 27)
GGAAGAAAAAGAATATCCCCAAAAGAGGAGTGACTTGGGATGGGGG

and it generated 4.9% as much light as the most common minimized catalytic core in the reselection (SEQ ID NO. 25).

The next construct in the series contains a A G:G triple at position 16-17-37 in the triple helix. It has the sequence

(SEQ ID NO. 118)
GGAAGAAAAAGAATATCCCAAAAAGGGGAGTGACTTGGGATGGGGG

and it generated 92.3% as much light as the most common minimized catalytic core in the reselection (SEQ ID NO. 25).

The next construct in the series contains a G G:G triple at position 16-17-37 in the triple helix. It has the sequence

(SEQ ID NO. 119)
GGAAGAAAAAGAATATCCCGAAAAGGGGAGTGACTTGGGATGGGGG

and it generated 32.1% as much light as the most common minimized catalytic core in the reselection (SEQ ID NO. 25).

The next construct in the series contains a T G:G triple at position 16-17-37 in the triple helix. It has the sequence

(SEQ ID NO. 120)
GGAAGAAAAAGAATATCCCTAAAAGGGGAGTGACTTGGGATGGGGG

and it generated 73.1% as much light as the most common minimized catalytic core in the reselection (SEQ ID NO. 25).

The next construct in the series contains a A A:A triple at position 16-17-37 in the triple helix. It has the sequence

(SEQ ID NO. 121)
GGAAGAAAAAGAATATCCCAAAAAAGGGAGTGACTTGGGATGGGAG

and it generated 59.3% as much light as the most common minimized catalytic core in the reselection (SEQ ID NO. 25).

The next construct in the series contains a C A:A triple at position 16-17-37 in the triple helix. It has the sequence

(SEQ ID NO. 122)
GGAAGAAAAAGAATATCCCCAAAAAGGGAGTGACTTGGGATGGGAG

and it generated 41.2% as much light as the most common minimized catalytic core in the reselection (SEQ ID NO. 25).

The next construct in the series contains a G A:A triple at position 16-17-37 in the triple helix. It has the sequence

(SEQ ID NO. 123)
GGAAGAAAAAGAATATCCCGAAAAAGGGAGTGACTTGGGATGGGAG

and it generated 26.9% as much light as the most common minimized catalytic core in the reselection (SEQ ID NO. 25).

The next construct in the series contains a A A:T triple at position 16-17-37 in the triple helix. It has the sequence

(SEQ ID NO. 124)
GGAAGAAAAAGAATATCCCAAAAAAGGGAGTGACTTGGGATGGGTG

and it generated 104.3% as much light as the most common minimized catalytic core in the reselection (SEQ ID NO. 25).

The next construct in the series contains a C A:T triple at position 16-17-37 in the triple helix. It has the sequence

(SEQ ID NO. 125)
GGAAGAAAAAGAATATCCCCAAAAAGGGAGTGACTTGGGATGGGTG

and it generated 107.4% as much light as the most common minimized catalytic core in the reselection (SEQ ID NO. 25).

The next construct in the series contains a G A:T triple at position 16-17-37 in the triple helix. It has the sequence

(SEQ ID NO. 126)
GGAAGAAAAAGAATATCCCGAAAAAGGGAGTGACTTGGGATGGGTG

and it generated 82.7% as much light as the most common minimized catalytic core in the reselection (SEQ ID NO. 25).

The next construct in the series contains a C-G A triple at position 16-17-37 in the triple helix. It has the sequence

(SEQ ID NO. 127)
GGAAGAAAAAGAATATCCCCAAAAGGGGAGTGACTTGGGATGGGAG

and it generated 50.9% as much light as the most common minimized catalytic core in the reselection (SEQ ID NO. 25).

The next construct in the series contains a C-G T triple at position 16-17-37 in the triple helix. It has the sequence

(SEQ ID NO. 128)
GGAAGAAAAAGAATATCCCCAAAAGGGGAGTGACTTGGGATGGGTG

and it generated 71% as much light as the most common minimized catalytic core in the reselection (SEQ ID NO. 25).

The next construct in the series contains a T-A G triple at position 16-17-37 in the triple helix. It has the sequence

(SEQ ID NO. 129)
GGAAGAAAAAGAATATCCCTAAAAAGGGAGTGACTTGGGATGGGGG

and it generated 29.6% as much light as the most common minimized catalytic core in the reselection (SEQ ID NO. 25).

The next construct in the series contains a A-T A triple at position 16-17-37 in the triple helix. It has the sequence

(SEQ ID NO. 130)
GGAAGAAAAAGAATATCCCAAAAATGGGAGTGACTTGGGATGGGAG

and it generated 89.1% as much light as the most common minimized catalytic core in the reselection (SEQ ID NO. 25).

The next construct in the series contains a G-C A triple at position 16-17-37 in the triple helix. It has the sequence

(SEQ ID NO. 131)
GGAAGAAAAAGAATATCCCGAAAACGGGAGTGACTTGGGATGGGAG

and it generated 38.7% as much light as the most common minimized catalytic core in the reselection (SEQ ID NO. 25).

The next construct in the series contains a T G T triple at position 16-17-37 in the triple helix. It has

(SEQ ID NO. 132)
GGAAGAAAAAGAATATCCCTAAAAGGGGAGTGACTTGGGATGGGTG

and it generated 35.2% as much light as the most common minimized catalytic core in the reselection (SEQ ID NO. 25).

The next construct in the series contains a G T A triple at position 16-17-37 in the triple helix. It has the sequence

(SEQ ID NO. 133)
GGAAGAAAAAGAATATCCCGAAAATGGGAGTGACTTGGGATGGGAG

and it generated 22.7% as much light as the most common minimized catalytic core in the reselection (SEQ ID NO. 25).

The next construct in the series contains a G G C triple at position 16-17-37 in the triple helix. It has the sequence

(SEQ ID NO. 134)
GGAAGAAAAAGAATATCCCGAAAAGGGGAGTGACTTGGGATGGGCG

and it generated 13.5% as much light as the most common minimized catalytic core in the reselection (SEQ ID NO. 25).

The next construct in the series contains a C C G triple at position 16-17-37 in the triple helix. It has the sequence

(SEQ ID NO. 135)
GGAAGAAAAAGAATATCCCCAAAACGGGAGTGACTTGGGATGGGGG

and it generated 7.2% as much light as the most common minimized catalytic core in the reselection (SEQ ID NO. 25).

The next construct in the series contains a C A G triple at position 16-17-37 in the triple helix. It has the sequence

(SEQ ID NO. 136)
GGAAGAAAAAGAATATCCCCAAAAAGGGAGTGACTTGGGATGGGGG

and it generated 11.3% as much light as the most common minimized catalytic core in the reselection (SEQ ID NO. 25).

### Example 12: Catalytic Activity of Minimized Catalytic Cores of Deoxyribozymes from the Reselection

The 50 most abundant minimized catalytic cores of deoxyribozymes in the reselection (cores 1-50) were tested as described in Example 4, except using 0.25 mM rather than 1 mM CDP-Star™ substrate. These correspond to sequences in which R¹ was replaced by AAAA, R² was replaced by AAAA, and R³ was deleted. Core 1 (SEQ ID NO. 25) is already described in Example 7, Cores 2 (SEQ ID NO:26) and 3 (SEQ ID NO:32) are already described in Example 8, Cores 8 (SEQ ID NO:40), 12 (SEQ ID NO:48), 19 (SEQ ID NO:55), 20 (SEQ ID NO:41), 21 (SEQ ID NO:42), 24 (SEQ ID NO:57), 39 (SEQ ID NO:47), 40 (SEQ ID NO:37), 42 (SEQ ID NO:46), and 48 (SEQ ID NO:56) are already described m Example 9. cores 16 (SEQ ID NO:66), 32 (SEQ ID NO:67), and 46 (SEQ ID NO:63) are already described in Example 10, and Cores 5 (SEQ ID NO:92) 6 (SEQ ID NO:108), 34 (SEQ ID NO:101), 41 (SEQ ID NO:94) are already described in Example 11. The remaining cores are listed here.

The sequence of core 4 is

(SEQ ID NO. 137)
GGAAGAAAAAGAATTTCCTCAAAAGGGGAGTGACTTGGGATGGGGG

and it generated 5.6% as much light as the most common minimized catalytic core in the reselection (SEQ ID NO. 25).

The sequence of core 7 is

(SEQ ID NO. 138)
GGCAGAAAAAGCATATCCCCAAAAGGGGAGTGACTTGGGATGGGGG

and it generated 8.2% as much light as the most common minimized catalytic core in the reselection (SEQ ID NO. 25).

The sequence of core 9 is

(SEQ ID NO. 139)
GGAAGAAAAAGCATATCCTCAAAAGGGGAGTGACTTGGGATGGGGG

and it generated 4% as much light as the most common minimized catalytic core in the reselection (SEQ ID NO. 25).

The sequence of core 10 is

(SEQ ID NO. 140)
GGAAGAAAAAGAATATCCGCAAAAGGGGAGTGACTTGGGATGGCGA

and it generated 4.4% as much light as the most common minimized catalytic core in the reselection (SEQ ID NO. 25).

The sequence of core 11 is

(SEQ ID NO. 141)
GGAAGAAAAAGATAGTAACCAAAAGGGGAGTGACTTGGGATGGGGG

and it generated 2.5% as much light as the most common minimized catalytic core in the reselection (SEQ ID NO. 25).

The sequence of core 13 is

(SEQ ID NO. 142)
GGAAGAAAAAGCATATCCCCAAAAGGGGAGTGACTTGGGATGGGGT

and it generated 6.2% as much light as the most common minimized catalytic core in the reselection (SEQ ID NO. 25).

The sequence of core 14 is

(SEQ ID NO. 143)
GGAAGAAAAAGAATATCCTCAAAAGGGGAGTGACTTGGGATGGGGT

and it generated 51.2% as much light as the most common minimized catalytic core in the reselection (SEQ ID NO. 25).

The sequence of core 15 is

(SEQ ID NO. 144)
GGAAGTAAAAGAATATCCTCAAAAGGGGAGTGACTTGGGATGGGGT

and it generated 36.1% as much light as the most common minimized catalytic core in the reselection (SEQ ID NO. 25).

The sequence of core 17 is

(SEQ ID NO. 145)
GGAAGAAAAAGTATATCCTCAAAAGGGGAGTGACTTGGGATGGGGG

and it generated 14.4% as much light as the most common minimized catalytic core in the reselection (SEQ ID NO. 25).

The sequence of core 18 is

(SEQ ID NO. 146)
GGAAGAAAAAGCATATCCACAAAAGGGGAGTGACTTGGGATGGGGG

and it generated 10.3% as much light as the most common minimized catalytic core in the reselection (SEQ ID NO. 25).

The sequence of core 22 is

(SEQ ID NO. 147)
GGCAGTAAAAGCATATCCACAAAAGGGGAGTGACTTGGGATGGGGG

and it generated 9.5% as much light as the most common minimized catalytic core in the reselection (SEQ ID NO. 25).

The sequence of core 23 is

(SEQ ID NO. 148)
GGAAGAAAAAGGATATCCTCAAAAGGGGAGTGACTTGGGATGGGGG

and it generated 14% as much light as the most common minimized catalytic core in the reselection (SEQ ID NO. 25).

The sequence of core 25 is

(SEQ ID NO. 149)
GGAAGTAAAAGCATATCCTCAAAAGGGGAGTGACTTGGGATGGGGG

and it generated 10.5% as much light as the most common minimized catalytic core in the reselection (SEQ ID NO. 25).

The sequence of core 26 is

(SEQ ID NO. 150)
GGAAGAAAAAGAATATCATCAAAAGGGGAGTGACTTGGGATGGGGG

and it generated 9.7% as much light as the most common minimized catalytic core in the reselection (SEQ ID NO. 25).

The sequence of core 27 is

(SEQ ID NO. 151)
GGAAGAAAAATATCTACCCTAAAAGGGGAGTGACTTGGGATGGGGG

and it generated 8.9% as much light as the most common minimized catalytic core in the reselection (SEQ ID NO. 25).

The sequence of core 28 is

(SEQ ID NO. 152)
GGAAGAAAAAGCATATCCCCAAAAGGGGAGTGACTTGGGATGAGGG

and it generated 12.7% as much light as the most common minimized catalytic core in the reselection (SEQ ID NO. 25).

The sequence of core 29 is

(SEQ ID NO. 153)
GGAAGAAAAAGAATATCCTCAAAAGGGGAGTGACTTGGGATAGGGG

and it generated 21.5% as much light as the most common minimized catalytic core in the reselection (SEQ ID NO. 25).

The sequence of core 30 is

(SEQ ID NO. 154)
GGAAGAAAAAGGATATCCACAAAAGGGGAGTGACTTGGGATGGGGG

and it generated 21.1% as much light as the most common minimized catalytic core in the reselection (SEQ ID NO. 25).

The sequence of core 31 is

(SEQ ID NO. 155)
GGAAGAAAAAGCATATCCTCAAAAGAGGAGTGACTTGGGATGGTGG

and it generated 13.5% as much light as the most common minimized catalytic core in the reselection (SEQ ID NO. 25).

The sequence of core 33 is

(SEQ ID NO. 156)
GGCAGAAAAAGAATATCCTCAAAAGGGGAGTGACTTGGGATGGGGG

and it generated 121.6% as much light as the most common minimized catalytic core in the reselection (SEQ ID NO. 25).

The sequence of core 33 is

(SEQ ID NO. 157)
GGAAGTAAAAGAATATCACCAAAAGGGGAGTGACTTGGGATGGGGG

and it generated 50.2% as much light as the most common minimized catalytic core in the reselection (SEQ ID NO. 25).

The sequence of core 36 is

(SEQ ID NO. 158)
GGAAGAAAAAGAACATCCTCAAAAGGGGAGTGACTTGGGATGGGGG

and it generated 27.4% as much light as the most common minimized catalytic core in the reselection (SEQ ID NO. 25).

The sequence of core 37 is

(SEQ ID NO. 159)
GGAAGAAAAAGCATATACCCAAAAGGGGAGTGACTTGGGATGGGGG

and it generated 14.5% as much light as the most common minimized catalytic core in the reselection (SEQ ID NO. 25).

The sequence of core 38 is

(SEQ ID NO. 160)
GGAAGAAAAAGCGTATACCCAAAAGGGGAGTGACTTGGGATGGGGC

and it generated 11.9% as much light as the most common minimized catalytic core in the reselection (SEQ ID NO. 25).

The sequence of core 43 is

(SEQ ID NO. 161)
GGAAGTAAAAGAATTTCCCCAAAAGGGGAGTGACTTGGGATGGGGG

and it generated 71.5% as much light as the most common minimized catalytic core in the reselection (SEQ ID NO. 25).

The sequence of core 44 is

(SEQ ID NO. 162)
GGAAGAAAAAGAATATCCTCAAAAGGGGAGTGACTTGGGATGGGGA

and it generated 44.5% as much light as the most common minimized catalytic core in the reselection (SEQ ID NO. 25).

The sequence of core 45 is

(SEQ ID NO. 163)
GGAAGAAAAAGAATATCCTTAAAAGAGGAGTGACTTGGGATGGTGG

and it generated 60.4% as much light as the most common minimized catalytic core in the reselection (SEQ ID NO. 25).

The sequence of core 47 is

(SEQ ID NO. 164)
GGCAGAAAAAGAATATCCCCAAAAGGGGAATGACTTGGGATGGGGG

and it generated 23.9% as much light as the most common minimized catalytic core in the reselection (SEQ ID NO. 25).

The sequence of core 49 is

(SEQ ID NO. 165)
GGAAGAAAAAGAATATTCTCAAAAGGGGAGTGACTTGGGATGAGGG

and it generated 8.2% as much light as the most common minimized catalytic core in the reselection (SEQ ID NO. 25).

The sequence of core 50 is

(SEQ ID NO. 166)
GGAAGAAAAAGAATTTCCCCAAAAGGGAAGTGACTTGGGATTGGGG

and it generated 8.5% as much light as the most common minimized catalytic core in the reselection (SEQ ID NO. 25).

### Example 13: Identification of Sites in the Deoxyribozyme at Which New Sequences Can Be Inserted

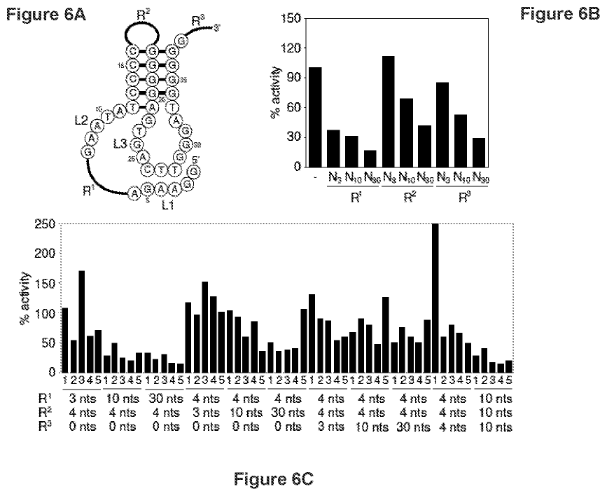

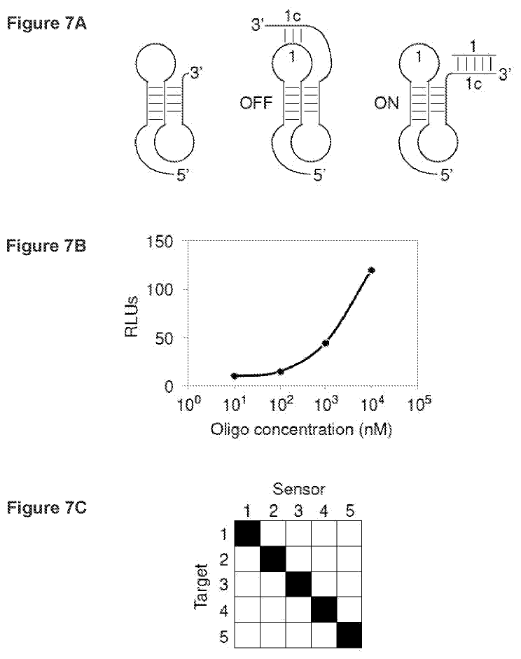

Deletion analysis (described in Example 7) showed that R¹ (originally 26 nucleotides) could be replaced by AAAA, that R¹ (originally 18 nucleotides) could be replaced by AAAA, and R³ (originally 18 nucleotides) could be deleted (see FIG. 6 for the locations of R¹, R², and R³ mapped onto the secondary structure of the deoxyribozyme). To determine the extent to which other nucleotides could also be inserted at these positions, a series of constructs was generated in which random sequence regions of different lengths (N=25% A, 25% C, 25% G, and 25% T) were inserted at R¹, R³. Deoxyribozymes were tested as described in Example 4, except using 0.25 mM rather than 1 mM CDP-Star™ substrate. The notation N₃, N₁₀, N₃₀ means that the oligonucleotide tested contained a mix of sequences at the positions indicated with Ns—a particular oligonucleotide would have in each position a 25% chance of having an A, a 25% chance of having a C, a 25% chance of haying G, and a 25% chance of having a T. This allows us to conclude that the sequence works regardless of the specific nucleotides in these positions.

The first deoxyribozyme in the series

(SEQ ID NO. 167)
GGAAGAN₃GAATATCCCCAAAAGGGGAGTGACTTGGGATGGGGG

contained an N3 insertion at R¹. This generated 36.8% as much light as SEQ ID NO. 25 (a variant with an AAAA insertion at R¹).

The second deoxyribozyme in the series

(SEQ ID NO. 168)
GGAAGAN₁₀GAATATCCCCAAAAGGGGAGTGACTTGGGATGGGGG

contained an N10 insertion at R¹. This generated 31.6% as much light as SEQ ID NO. 25 (a variant with an AAAA insertion at R¹).

The third deoxyribozyme in the series

(SEQ ID NO. 169)
GGAAGAN₃₀GAATATCCCCAAAAGGGGAGTGACTTGGGATGGGGG

contained an N30 insertion at R¹. This generated 16.5% as much light as SEQ ID NO. 25 (a variant with AAAA insertion at R¹).

The fourth deoxyribozyme in the series

(SEQ ID NO. 170)
GGAAGAAAAAGAATATCCCCN₃GGGGAGTGACTTGGGATGGGGG

contained an N3 insertion at R². This generated 110.8% as much light as SEQ ID NO. 25 (a variant with an AAAA insertion at R²).

The fifth deoxyribozyme in the series

(SEQ ID NO. 171)
GGAAGAAAAAGAATATCCCCN₁₀GGGGAGTGACTTGGGATGGGGG

contained an N10 insertion at R². This generated 68.4% as much light as SEQ ID NO. 25 (a variant with an AAAA insertion at R²).

The sixth deoxyribozyme in the series

(SEQ ID NO. 172)
GGAAGAAAAAGAATATCCCCN₃₀GGGGAGTGACTTGGGATGGGGG

contained an N30 insertion at R². This generated 41.1% as much light as SEQ ID NO. 25 (a variant with an AAAA insertion at R²).

The seventh deoxyribozyme in the series

(SEQ ID NO. 173)
GGAAGAAAAAGAATATCCCCAAAAGGGGAGTGACTTGGGATGGGGGN₃

contained an N3 extension at at R³. This generated 84.5% as much light as SEQ ID NO. 25 (a variant with no extension at R³).

The eighth deoxyribozyme in the series

(SEQ ID NO. 174)
GGAAGAAAAAGAATATCCCCAAAAGGGGAGTGACTTGGGATGGGGGN₁₀

contained an N10 extension at R³. This generated 52% as much light as SEQ ID NO. 25 (a variant with no extension at R³).

The ninth deoxyribozyme in the series

(SEQ ID NO. 175)
GGAAGAAAAAGAATATCCCCAAAAGGGGAGTGACTTGGGATGGGGGN₃₀

contained an N30 extension at R³. This generated 29.1% as much light as SEQ ID NO. 25 (a variant with no extension at R³).

### Example 14: Further Characterization of Insertion Sites in the Deoxyribozyme

To further characterize deoxyribozyme insertion sites, five different variants of each of the nine R¹-R²-R³ architectures described in Example 13 were generated in which arbitrary sequences were inserted at R¹, R², and/or R³. In addition, five different variants of each of two new R¹-R²-R³ architectures were generated. See FIG. 6 for the locations of R¹, R², and R³ mapped onto the secondary structure of the deoxyribozyme. Deoxyribozymes were tested as described in Example 4, except using 0.25 mM rather than 1 mM CDP-Star™ substrate.

The first deoxyribozyme in the series

(SEQ ID NO. 176)
GGAAGAAGAGAATATCCCCAAAAGGGGAGTGACTTGGGATGGGGG

contained an arbitrary 3 nucleotide sequence at R¹, AAAA at R², and no nucleotides at R³. This generated 108.2% as much light as SEQ ID NO. 25 (a variant with the sequence AAAA at R¹, AAAA at R², and no nucleotides at R³).

The next deoxyribozyme in the series

(SEQ ID NO. 177)
GGAAGAACTGAATATCCCCAAAAGGGGAGTGACTTGGGATGGGGG

contained an arbitrary 3 nucleotide sequence at R¹, AAAA at R², and no nucleotides at R³. This generated 54.3% as much light as SEQ ID NO. 25.

The next deoxyribozyme in the series

(SEQ ID NO. 178)
GGAAGATAAGAATATCCCCAAAAGGGGAGTGACTTGGGATGGGGG

contained an arbitrary 3 nucleotide sequence at R¹, AAAA at R², and no nucleotides at R³. This generated 169.1% as much light as SEQ ID NO. 25.

The next deoxyribozyme in the series

(SEQ ID NO. 179)
GGAAGAACCGAATATCCCCAAAAGGGGAGTGACTTGGGATGGGGG

contained an arbitrary 3 nucleotide sequence at R¹, AAAA at R², and no nucleotides at R³. This generated 61.5% as much light as SEQ ID NO. 25.

The next deoxyribozyme in the series

(SEQ ID NO. 180)
GGAAGACCAGAATATCCCCAAAAGGGGAGTGACTTGGGATG
GGGG

contained an arbitrary 3 nucleotide sequence at R¹, AAAA at R², and no nucleotides at R³. This generated 70.8% as much light as SEQ ID NO. 25.

The next deoxyribozyme in the series

(SEQ ID NO. 181)
GGAAGAGGGATGACCAGAATATCCCCAAAAGGGGAGTGACT
TGGGATGGGGG

contained an arbitrary 10 nucleotide sequence at R¹, AAAA at R², and no nucleotides at R³. This generated 29.6% as much light as SEQ ID NO. 25.

The next deoxyribozyme in the series

(SEQ ID NO. 182)
GGAAGAACGTAATCGGGAATATCCCCAAAAGGGGAGTGACT
TGGGATGGGGG

contained an arbitrary 10 nucleotide sequence at R¹, AAAA at R², and no nucleotides at R³. This generated 49.1% as much light as SEQ ID NO. 25.

The next deoxyribozyme in the series

(SEQ ID NO. 183)
GGAAGACCTCGCTACCGAATATCCCCAAAAGGGGAGTGACT
TGGGATGGGGG

contained an arbitrary 10 nucleotide sequence at R¹, AAAA at R², and no nucleotides at R³. This generated 25.3% as much light as SEQ ID NO. 25.

The next deoxyribozyme in the series

(SEQ ID NO. 184)
GGAAGAGGGGGATCTTGAATATCCCCAAAAGGGGAGTGACT
TGGGATGGGGG

contained an arbitrary 10 nucleotide sequence at R¹, AAAA at R², and no nucleotides at R³. This generated 19.3% as much light as SEQ ID NO. 25.

The next deoxyribozyme in the series

(SEQ ID NO. 185)
GGAAGATTAACGTCTCGAATATCCCCAAAAGGGGAGTGACT
TGGGATGGGGG

contained an arbitrary 10 nucleotide sequence at R¹, AAAA at R², and no nucleotides at R³. This generated 33.3% as much light as SEQ ID NO. 25.

The next deoxyribozyme in the series

(SEQ ID NO. 186)
GGAAGACGCCCGCTGTTTTACAGTGCTGCAAAAAACGAATA
TCCCCAAAAGGGGAGTGACTTGGGATGGGGG

contained an arbitrary 30 nucleotide sequence at R¹, AAAA at R², and no nucleotides at R³. This generated 32.7% as much light as SEQ ID NO. 25.

The next deoxyribozyme in the series

(SEQ ID NO. 187)
GGAAGAAGTCTGGCCGTCGAATCTGGCCTATGTATTGAATA
TCCCCAAAAGGGGAGTGACTTGGGATGGGGG

contained an arbitrary 30 nucleotide sequence at R¹, AAAA at R², and no nucleotides at R³. This generated 22.5% as much light as SEQ ID NO. 25.

The next deoxyribozyme in the series

(SEQ ID NO. 188)
GGAAGACATAAGAGTGGTGGCGACCGTTACAATTAGGAATA
TCCCCAAAAGGGGAGTGACTTGGGATGGGGG

contained an arbitrary 30 nucleotide sequence at R¹, AAAA at R², and no nucleotides at R³. This generated 29.8% as much light as SEQ ID NO. 25.

The next deoxyribozyme in the series

(SEQ ID NO. 189)
GGAAGAAATGGGCTCGAATTAACGGGTAGTTAATTAGAATA
TCCCCAAAAGGGGAGTGACTTGGGATGGGGG

contained an arbitrary 30 nucleotide sequence at R¹, AAAA at R², and no nucleotides at R³. This generated 15.7% as much light as SEQ ID NO. 25.

The next deoxyribozyme in the series

(SEQ ID NO. 190)
GGAAGAAGGTGATAGGGGAGCGCACGGAATTACCCAGAATA
TCCCCAAAAGGGGAGTGACTTGGGATGGGGG

contained an arbitrary 30 nucleotide sequence at R¹, AAAA at R², and no nucleotides at R³. This generated 14.9% as much light as SEQ ID NO. 25.

The next deoxyribozyme in the series

(SEQ ID NO. 191)
GGAAGAAAAAGAATATCCCCGGAGGGGAGTGACTTGGGATGGGGG

contained AAAA at R¹, an arbitrary 3 nucleotide sequence at R², and no nucleotides at R³. This generated 116.5% as much light as SEQ ID NO. 25.

The next deoxyribozyme in the series

(SEQ ID NO. 192)
GGAAGAAAAAGAATATCCCCGAGGGGGAGTGACTTGGGATGGGGG

contained AAAA at R¹, an arbitrary 3 nucleotide sequence at R², and no nucleotides at R³. This generated 96.7% as much light as SEQ ID NO. 25.

The next deoxyribozyme in the series

(SEQ ID NO. 193)
GGAAGAAAAAGAATATCCCCTAAGGGGAGTGACTTGGGATGGGGG

contained AAAA at R¹, an arbitrary 3 nucleotide sequence at R², and no nucleotides at R³. This generated 151.2% as much light as SEQ ID NO. 25.

The next deoxyribozyme in the series

(SEQ ID NO. 194)
GGAAGAAAAAGAATATCCCCGCTGGGGAGTGACTTGGGATGGGGG

contained AAAA at R¹, an arbitrary 3 nucleotide sequence at R², and no nucleotides at R³. This generated 127.9% as much light as SEQ ID NO. 25.

The next deoxyribozyme in the series

(SEQ ID NO. 195)
GGAAGAAAAAGAATATCCCCAATGGGGAGTGACTTGGGATGGGGG

contained AAAA at R¹, an arbitrary 3 nucleotide sequence at R², and no nucleotides at R³. This generated 101.9% as much light as SEQ ID NO. 25.

The next deoxyribozyme in the series

(SEQ ID NO. 196)
GGAAGAAAAAGAATATCCCCGGGAAGGCCTGGGGAGTGACTTGGG
ATGGGGG

contained AAAA at R¹, an arbitrary 10 nucleotide sequence at R², and no nucleotides at R³. This generated 104% as much light as SEQ ID NO. 25.

The next deoxyribozyme in the series

(SEQ ID NO. 197)
GGAAGAAAAAGAATATCCCCTTTTACTGTGGGGGAGTGACTTGGG
ATGGGGG

contained AAAA at R¹, an arbitrary 10 nucleotide sequence at R², and no nucleotides at R³. This generated 92.8% as much light as SEQ ID NO. 25.

The next deoxyribozyme in the series

(SEQ ID NO. 198)
GGAAGAAAAAGAATATCCCCCTTAGCAAATGGGGAGTGACTTGGG
ATGGGGG

contained AAAA at R¹, an arbitrary 10 nucleotide sequence at R², and no nucleotides at R³. This generated 59.9% as much light as SEQ ID NO. 25.

The next deoxyribozyme in the series

(SEQ ID NO. 199)
GGAAGAAAAAGAATATCCCCAAAAAGCATGGGGGAGTGACTTGGG
ATGGGGG

contained AAAA at R¹, an arbitrary 10 nucleotide sequence at R², and no nucleotides at R³. This generated 86% as much light as SEQ ID NO. 25.

The next deoxyribozyme in the series

(SEQ ID NO. 200)
GGAAGAAAAAGAATATCCCCAATTTCACCCGGGGAGTGACTTGGG
ATGGGGG

contained AAAA at R¹, an arbitrary 10 nucleotide sequence at R², and no nucleotides at R³. This generated 36.2% as much light as SEQ ID NO. 25.

The next deoxyribozyme in the series

(SEQ ID NO. 201)
GGAAGAAAAAGAATATCCCCTAACGAGTCTCAAAAGCTACTTCGC
TATAAGGGGAGTGACTTGGGATGGGGG

contained AAAA at R¹, an arbitrary 30 nucleotide sequence at R², and no nucleotides at R³. This generated 50.4% as much light as SEQ ID NO. 25.

The next deoxyribozyme in the series

(SEQ ID NO. 202)
GGAAGAAAAAGAATATCCCCGGGTGACACCACCGATAACGAGCCT
TAAACGGGGAGTGACTTGGGATGGGGG

contained AAAA at R¹, an arbitrary 30 nucleotide sequence at R², and no nucleotides at R³. This generated 35.7% as much light as SEQ ID NO. 25.

The next deoxyribozyme in the series

(SEQ ID NO. 203)
GGAAGAAAAAGAATATCCCCCGACCACCAACCCAAGTAGACATTA
TGAGTGGGGAGTGACTTGGGATGGGGG

contained AAAA at R¹, an arbitrary 30 nucleotide sequence at R², and no nucleotides at R³. This generated 38.1% as much light as SEQ ID NO. 25.

The next deoxyribozyme in the series

(SEQ ID NO. 204)
GGAAGAAAAAGAATATCCCCTGGCTCCCTCCAAGGGTAGGCATTC
ACGAGGGGGAGTGACTTGGGATGGGGG

contained AAAA at R¹, an arbitrary 30 nucleotide sequence at R², and no nucleotides at R³. This generated 41% as much light as SEQ ID NO. 25.

The next deoxyribozyme in the series

(SEQ ID NO. 205)
GGAAGAAAAAGAATATCCCCGCAGTGTAGTGAGAGGACCTCAGCA
CACGCGGGGAGTGACTTGGGATGGGGG

contained AAAA at R¹, an arbitrary 30 nucleotide sequence at R², and no nucleotides at R³. This generated 106.2% as much light as SEQ NO. 25.

The next deoxyribozyme in the series

(SEQ ID NO. 206)
GGAAGAAAAAGAATATCCCCAAAAGGGGAGTGACTTGGGATGGGG
GGAT

contained AAAA at R¹, AAAA at R², and an arbitrary 3 nucleotide sequence at R³. This generated 131% as much light as SEQ ID NO. 25.

The next deoxyribozyme in the series

(SEQ ID NO. 207)
GGAAGAAAAAGAATATCCCCAAAAGGGGAGTGACTTGGGATGGGG
GTGG

contained AAAA at R¹, AAAA at R², and an arbitrary 3 nucleotide sequence at R³. This generated 90.4% as much light as SEQ ID NO. 25.

The next deoxyribozyme in the series

(SEQ ID NO. 208)
GGAAGAAAAAGAATATCCCCAAAAGGGGAGTGACTTGGGATGGGG
GTGT

contained AAAA at R¹, AAAA at R², and an arbitrary 3 nucleotide sequence at R³. This generated 86.3% as much light as SEQ ID NO. 25.

The next deoxyribozyme in the series

(SEQ ID NO. 209)
GGAAGAAAAAGAATATCCCCAAAAGGGGAGTGACTTGGGATGGGG
GGCG

contained AAAA at R¹, AAAA at R², and an arbitrary 3 nucleotide sequence at R³. This generated 53.4% as much light as SEQ ID NO. 25.

The next deoxyribozyme in the series

(SEQ ID NO. 210)
GGAAGAAAAAGAATATCCCCAAAAGGGGAGTGACTTGGGAT
GGGGGTGG

contained AAAA at R¹, AAAA at R², and an arbitrary 3 nucleotide sequence at R³. This generated 61.1% as much light as SEQ ID NO. 25. The next deoxyribozyme in the series

(SEQ ID NO. 211)
GGAAGAAAAAGAATATCCCCAAAAGGGGAGTGACTTGGGA
TGGGGGACCTAGGCAA

contained AAAA at R¹, AAAA at R², and an arbitrary 10 nucleotide sequence at R³. This generated 66.9% as much light as SEQ ID NO. 25.

The next deoxyribozyme in the series

(SEQ ID NO. 212)
GGAAGAAAAAGAATATCCCCAAAAGGGGAGTGACTTGGGA
TGGGGGTGTTCATCAA

contained AAAA at R¹, AAAA at R², and an arbitrary 10 nucleotide sequence at R³. This generated 90.9% as much light as SEQ ID NO. 25.

The next deoxyribozyme in the series

(SEQ ID NO. 213)
GGAAGAAAAAGAATATCCCCAAAAGGGGAGTGACTTGGGA
TGGGGGGATAGACTCT

contained AAAA at R¹, AAAA at R², and an arbitrary 10 nucleotide sequence at R³. This generated 79.8% as much light as SEQ ID NO. 25.

The next deoxyribozyme in the series

(SEQ ID NO. 214)
GGAAGAAAAAGAATATCCCCAAAAGGGGAGTGACTTGGGA
TGGGGGATACCCTTGT

contained AAAA at R¹, AAAA at R², and an arbitrary 10 nucleotide sequence at R³. This generated 47% as much light as SEQ ID NO. 25.

The next deoxyribozyme in the series

(SEQ ID NO. 215)
GGAAGAAAAAGAATATCCCCAAAAGGGGAGTGACTTGGGA
TGGGGGCGATCTGACG

contained AAAA at R¹, AAAA at R², and an arbitrary 10 nucleotide sequence at R³. This generated 125.8% as much light as SEQ ID NO. 25.

The next deoxyribozyme in the series

(SEQ ID NO. 216)
GGAAGAAAAAGAATATCCCCAAAAGGGGAGTGACTTGGGA
TGGGGGCGAAGTTGGCAAACATTATAAGTTGGGGAG

contained AAAA at R¹, AAAA at R², and an arbitrary 30 nucleotide sequence at R³. This generated 49.5% as much light as SEQ ID NO. 25.

The next deoxyribozyme in the series

(SEQ ID NO. 217)
GGAAGAAAAAGAATATCCCCAAAAGGGGAGTGACTTGGGA
TGGGGGCAAGGACTTTATACAATGCTCGCGGGTCGA

contained AAAA at R¹, AAAA at R², and an arbitrary 30 nucleotide sequence at R³. This generated 76.2% as much light as SEQ ID NO. 25.

The next deoxyribozyme in the series

(SEQ ID NO. 218)
GGAAGAAAAAGAATATCCCCAAAAGGGGAGTGACTTGGGA
TGGGGGTGGAGCATGCATTCTTGGACAAAATCTTGT

contained AAAA at R¹, AAAA at R², and an arbitrary 30 nucleotide sequence at R³. This generated 61% as much light as SEQ ID NO. 25.

The next deoxyribozyme in the series

(SEQ ID NO. 219)
GGAAGAAAAAGAATATCCCCAAAAGGGGAGTGACTTGGGA
TGGGGGGTGCTACTTAAATTCTGTGTGTGACTTTAA

contained AAAA at R¹, AAAA at R², and an arbitrary 30 nucleotide sequence at R³. This generated 49.6% as much light as SEQ ID NO. 25.

The next deoxyribozyme in the series

(SEQ ID NO. 220)
GGAAGAAAAAGAATATCCCCAAAAGGGGAGTGACTTGGGA
TGGGGGTTGGCTGCCGTCCCAGGCGGCACGATAGAC

contained AAAA at R¹; AAAA at R², and an arbitrary 30 nucleotide sequence at R³. This generated 88.7% as much light as SEQ ID NO. 25.

The next deoxyribozyme in the series

(SEQ ID NO. 221)
GGAAGATCCTGAATATCCCCTTGAGGGGAGTGACTTGGGA
TGGGGGCTAG

contained an arbitrary 4 nucleotide sequence at R¹, R², and R³. This generated 252.4% as much light as SEQ ID NO. 25.

The next deoxyribozyme in the series

(SEQ ID NO. 222)
GGAAGAGAACGAATATCCCCACCTGGGGAGTGACTTGGGA
TGGGGGCGAA

contained an arbitrary 4 nucleotide sequence at R¹, R², and R³. This generated 59.9% as much light as SEQ ID NO. 25.

The next deoxyribozyme in the series

(SEQ ID NO. 223)
GGAAGATGCCGAATATCCCCGTCGGGGGAGTGACTTGGGA
TGGGGGTACG

contained an arbitrary 4 nucleotide sequence at R¹, R², and R³. This generated 78.7% as much light as SEQ ID NO. 25.

The next deoxyribozyme in the series

(SEQ ID NO. 224)
GGAAGAGCGAGAATATCCCCACAGGGGGAGTGACTTGGGA
TGGGGGACGG

contained an arbitrary 4 nucleotide sequence at R¹, R², and R³. This generated 66.1% as much light as SEQ ID NO. 25.

The next deoxyribozyme in the series

(SEQ ID NO. 225)
GGAAGACCCTGAATATCCCCCCGTGGGGAGTGACTTGGGATGGGGGGCT
C

contained an arbitrary 4 nucleotide sequence at R¹, R², and R³. This generated 48.7% as much light as SEQ ID NO. 25.

The next deoxyribozyme in the series

(SEQ ID NO. 226)
GGAAGACTTTACCTACGAATATCCCCGTAGGCACTCGGGGAGTGACTTG
GGATGGGGGACGCTCCCAT

contained an arbitrary 10 nucleotide sequence at R¹, R², and R³. This generated 28.2% as much light as SEQ ID NO. 25.

The next deoxyribozyme in the series

(SEQ ID NO. 227)
GGAAGATCGATGAGGCGAATATCCCCAGATCACCTAGGGGAGTGACTTG
GGATGGGGGAGCGGCACCG

contained an arbitrary 10 nucleotide sequence at R¹, R², and R². This generated 40.7% as much light as SEQ ID NO. 25.

The next deoxyribozyme in the series

(SEQ ID NO. 228)
GGAAGACTCCTITTATGAATATCCCCCTGTCTTTAAGGGGAGTGACTTG
GGATGGGGGCAGATCGCTA

contained an arbitrary 10 nucleotide sequence at R¹, R², and R³. This generated 17.2% as much light as SEQ ID NO. 25.

The next deoxyribozyme in the series

(SEQ ID NO. 229)
GGAAGATCGACCGCCAGAATATCCCCTCAGCGCGAGGGGGAGTGACTTG
GGATGGGGGAGAAGGAAGT

contained an arbitrary 10 nucleotide sequence at R¹, R², and R³. This generated 15% as much light as SEQ ID NO. 25.

The next deoxyribozyme in the series

(SEQ ID NO. 230)
GGAAGATAACGTAGGGGAATATCCCCCCAGAAGGTAGGGGAGTGACTTG
GGATGGGGGCCCCGGACGT

contained an arbitrary 10 nucleotide sequence at R¹, R², and R³. This generated 19.8% as much light SEQ ID NO. 25.

### Example 15: Method for Detection of Specific Oligonucleotide Sequences using a Light-Producing Deoxyribozyme Sensor

To generate a version of this deoxyribozyme that can sense oligonucleotides with specific sequences, we inserted part of the sequence of the target oligonucleotide into R², and the reverse complement of the full target oligonucleotide into R³. In the absence of the target, R² and R³ form a helix by Watson-Clack interactions that inhibits the deoxyribozyme. In the presence of the target, however, the inhibitory helix cannot form because R³ binds to the target rather than R². This results in catalytic activity and the production of light. Deoxyribozymes were tested as described, in Example 4 (except using 0.25 mM rather than 1 mM CDP-Star™ substrate) in the presence of either 0 μM or 10 μM oligonucleotide ligand (=target).

The first sensor we generated

(SEQ ID NO. 231)
GGAAGAAAAAGAATATCCCCCAATCGTGCGGGGAGTGACTTGGGATGGG
GGAAGCACGATTGACCTTC (target is GAAGGTCAATCGTGC,
SEQ ID NO. 232)

generated light with a rate enhancement of 38.4 -fold in the presence of 10 μM target and 1.9-fold in the absence of the target.

The second sensor we generated

(SEQ ID NO. 233)
GGAAGAAAAAGAATATCCCCCTCTTAAGAGGGGAGTGACTTGGGATGGG
GGAATCTTAAGAGAAGGGA (target is TCCCTTCTCTTAAGA,
SEQ ID NO. 234)

generated light with a rate enhancement of 15-fold in the presence of 10 μM target and 2.7-fold in the absence of the target.

The third sensor we generated

(SEQ ID NO. 235)
GGAAGAAAAAGAATATCCCCGGCACTGATGGGGAGTGACTTGGGATGGG
GGAAATCAGTGCCCAGTGG (target is CCACTGGGCACTGAT,
SEQ ID NO. 236)

generated light with a rate enhancement of 35.6-fold relative to the reaction in the absence of deoxyribozyme in the presence of 10 μM target and 2.4-fold in the absence of the target.

The fourth sensor we generated

(SEQ ID NO. 237)
GGAAGAAAAAGAATATCCCCATGATCGGAGGGGAGTGACTTGGGATGGG
GGAATCCGATCATCCGAAG (target is CTTCGGATGATCGGA,
SEQ ID NO. 238)

generated light with a rate enhancement of 44.8-fold relative to the reaction in the absence of deoxyribozyme in the presence of 10 μM target and 6.8-fold in the absence of the target.

The fifth sensor we generated

(SEQ ID NO. 239)
GGAAGAAAAAGAATATCCCCAGTAATAGCGGGGAGTGACTTGGGATGGG
GGAAGCTATTACTTATCTT (target is AAGATAAGTAATAGC,
SEQ ID NO. 240)

generated light with a rate enhancement of 37.5-fold relative to the reaction in the absence of deoxyribozyme in the presence of 10 μM target and 4.6-fold in the absence of the target.

### Example 16: Effect of Reaction Conditions on Deoxyribozyme-Catalyzed Light Production

A deoxyribozyme with the sequence SEQ ID NO. 25 was tested as described in Example 4 except for the following changes.

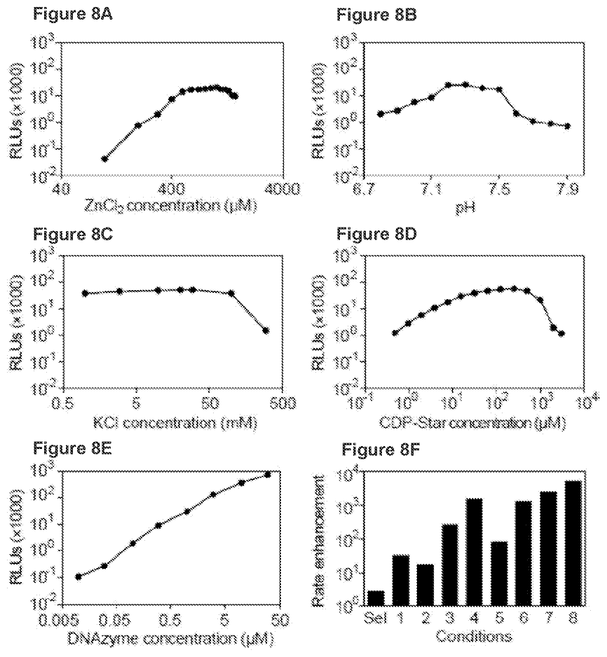

In FIG. 8A, the buffer contained 1 μM deoxyribozyme, 20 mM HEPES, pH 7.4, 20 mM KCl, varying concentrations of ZnCl₂, and 62.5 μM CDP-Star™.

In FIG. 8B, the buffer contained 1 μM. deoxyribozyme, 20 mM HEPES at various pH values, 20 mM KCl, 0.65 mM ZnCl₂, and 62.5 μM CDP-Star™.

In FIG. 8C, the buffer contained 1 μM deoxyribozyme, 20 mM HEPES, pH 7.4, varying concentrations of KCl, 1 nM ZnCl₂, and 62.5 μM CDP-Star™.

In FIG. 8D, the buffer contained 1 μM deoxyribozyme, 20 mM HEPES, pH 7.4, 20 mM KCl, 0.65 mM ZnCl₂, and varying concentrations of CDP-Star.

In FIG. 8E, the buffer contained varying concentrations of deoxyribozyme, 20 mM KCl, 0.65 mM ZnCl₂, 20 mM HEPES, pH 7.4, and 62.5 μM CDP-Star.

In FIG. 8F, the column labeled “Sel” indicates the amount of light produced using the conditions of the original selection (1 μM deoxyribozyme, 50 mM HEPES, pH 7.4, 200 mM KCl, 1 μM CeCl₃, 0.1 μM PbCl₂, 1 mM ZnCl₂, and 1000 μM CDP-Star).

All other reactions in FIG. 8F were performed in the absence of CeCl₃ and PbCl₂. Reactions contained either 20 mM (columns 2, 5, 6, and 8) or 200 mM (columns 1, 3, 4, and 7) KCl, 62.5 μM (columns 3, 5, 7, and 8) or 1000 nM (columns 1, 2 4, and 6) CDP-Star, 1 μM (columns 1, 2, 3, and 5) or 30 μM (columns 4, 6, 7, and 8) deoxyribozyme, 0.65 mM ZnCl₂ (columns 1-8), 15% DMSO (columns 1-8) and 20 mM HEPES, pH 7.4 (columns 1-8).

The y axis shows the rate enhancement of light production (the amount of light produced in the presence of deoxyribozyme divided by the amount of light produced in the absence of deoxyribozyme).

For all panels, points show the average of at least three experiments.

1. A deoxyribozyme containing or consisting of nucleotide sequence:
(SEQ ID NO. 1)
5′-X¹x²X³AX⁴X⁵-R¹-X⁶X⁷X⁸X⁹X¹⁰-bp1-helix1-R²-helix2-
bp2-X¹¹TGACX¹²TGGGAX¹³-helix3-X¹⁴-R³-3′
wherein
X¹ is G or T; X² is G or T; X³ is A or C; X⁴ is G or T; X⁵ is A or T; X⁶ is G or T or A; X⁷ is A or C or G or T;
X⁸ is A or G or T; X⁹ is T or C or A or G; X¹⁰ is A or T or G; X¹¹ is G or A; X¹² is T or C or G; X¹³ is T or G or C; X¹⁴ is G or A or C or T;
R¹ represents a nucleotide sequence containing 3 to 30 nucleotides;
R² represents a nucleotide sequence containing 3 to 30 nucleotides;
R³ represents a nucleotide sequence containing 0 to 30 nucleotides;
bp1 and bp2 are nucleotides selected from G, A, C, T, which together form a base pair;
helix1, helix2, and helix3 each independently denotes a nucleotide sequence preferably containing 4 or more nucleotides, wherein helix1, helix2, and helix3 together form a triple helical structure.

2. The deoxyribozyme according to claim 1, containing or consisting of nucleotide sequence:
(SEQ ID NO. 1)
5′-X¹x²X³AX⁴X⁵-R¹-X⁶X⁷X⁸X⁹X¹⁰-bp1-helix1-R²-helix2-
bp2-X¹¹TGACX¹²TGGGAX¹³-helix3-X¹⁴-R³-3′
wherein
X¹ is G; X² is G; X³ is A or C; X⁴ is G; X⁵ is A or T; X⁶ is G; X⁷ is A; X⁸ is A; X⁹ is T or C; X¹⁰ is A; X¹¹ is G;
X¹² is T or C; X¹³ is T; X¹⁴ is G or A or C or T;
and wherein one or more, preferably one or two, of X¹-X¹⁴ are replaced by the following nucleotides: X¹=T;
X²=T; X⁴=T; X⁶=T or A; X⁷=C or G or T; X⁸=G or T; X⁹=A or G; X¹⁰=T or G; X¹¹=A; X¹²=G; X¹³=G or C.

3. The deoxyribozyme according to claim 1, containing or consisting of nucleotide sequence:
(SEQ ID NO. 2)
5′-GGX³AGX⁵-R¹-GAAX⁹A-bp1-helix1-R²-helix2-bp2-
GTGACX¹²TGGGAT-helix3-X¹⁴-R³-3′
wherein
X³ is A or C; X⁵ is A or T; X⁹ is T or C; X¹² is T or C; X¹⁴ is G or A or C or T.

4. The deoxyribozyme according to claim 1, containing or consisting of nucleotide sequence:
(SEQ ID NO. 3)
5′-GGAAGA-R¹-GAATA-bp1-helix1-R²-helix2-bp2-
GTGACTTGGGAT-helix3-G-R³-3′,
wherein
R¹ represents a nucleotide sequence containing 3 to 30 nucleotides;
R² represents a nucleotide sequence containing 3 to 30 nucleotides;
R³ represents a nucleotide sequence containing 0 to 30 nucleotides;
bp1 and bp2 are nucleotides selected from G, A, C, T, which together form a base pair;
helix1, helix2, and helix3 each independently denotes a nucleotide sequence preferably containing 4 or more nucleotides, wherein helix1, helix2, and helix3 together form a triple helical structure.

5. The deoxyribozyme according to claim 1, wherein R³ is or contains an aptamer, and R² is or contains a sequence complementary to the aptamer.

6. The deoxyribozyme according to claim 1, wherein helix1, helix2, helix3 is CCCC, GGGG, GGGG; or CCCT, AGGG, GGGT; or CTCC, GGAG, GAGG; or CCCT, AGGG, GGGA.

7. The deoxyribozyme according to claim 1, containing or consisting of a sequence selected from the group consisting of SEQ ID NO:8, SEQ ID NO:15, SEQ ID NO:16, SEQ ID NO: 17, SEQ ID NO:18, SEQ ID NO:19, SEQ ID NO. 20, SEQ ID NO, 21, SEQ ID NO:22, SEQ ID NO. 23, SEQ ID NO:24, SEQ ID NO:25, SEQ ID NO:26, SEQ ID NO:28, SEQ ID NO:29, SEQ ID NO:32, SEQ ID NO:34, SEQ ID NO:35, SEQ ID NO:36, SEQ ID NO:37, SEQ ID NO:38, SEQ ID NO:40, SEQ ID NO:41, SEQ ID NO:42. SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ Ii) NO:61, SEQ NO:62, SEQ NO:63, SEQ ID NO:64, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:76, SEQ ID NO:77, SEQ ID NO: 78, SEQ ID NO:79, SEQ ID NO:80, SEQ ID NO:81, SEQ ID NO:82, SEQ ID NO:84, SEQ ID NO:92, SEQ ID NO:93, SEQ ID NO:94, SEQ ID NO:95, SEQ ID NO:96, SEQ ID NO:97, SEQ ID NO:98, SEQ ID NO:100, SEQ ID NO:101, SEQ ID NO:102, SEQ ID NO:108, SEQ ID NO:109, SEQ ID NO:110, SEQ ID NO:111, SEQ ID NO:113, SEQ ID NO:114, SEQ ID NO:115, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ I NO:128, SEQ ID NO:129, SEQ ID NO:130, SEQ ID NO:131, SEQ ID NO. 132, SEQ ID NO:133, SEQ ID NO:134, SEQ ID NO:136, SEQ ID NO:143, SEQ ID NO:144, SEQ ID NO:145, SEQ ID NO:146, SEQ ID NO:148, SEQ ID NO:149, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ NO:162, SEQ ID NO:163, SEQ ID NO:164, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:233, SEQ ID NO:235, SEQ ID NO:237, and SEQ ID NO:239.

8. A method of in vitro detection of the deoxyribozyme according to claim 1 as a signaling component of a molecular device in biosensing, diagnostics, and/or molecular computing.

9. The method of in vitro detection of a chemiluminescent substrate, containing a phosphate functional group, in a sample, containing the steps of:
bringing the sample into contact with the deoxyribozyme according to claim 1;
causing the generation of an optical signal by cleavage of the phosphate functional group from the substrate present in the sample by self-phosphorylation of the deoxyribozyme; and
detecting the generated optical signal.

10. The method of in vitro detection of the deoxyribozyme according to claim 1 in a sample, containing the steps of:
bringing the sample into contact with a chemiluminescent substrate containing a phosphate functional group;
causing the generation of an optical signal by cleavage of the phosphate functional group from the substrate by self-phosphorylation of the deoxyribozyme present in the sample; and
detecting the generated optical signal.

11. The method of in vitro detection of a ligand in a sample, containing the steps of:
bringing the sample into contact with the deoxyribozyme according to claim 1 and with a chemiluminescent substrate containing a phosphate functional group and capable of generating an optical signal upon cleavage of the phosphate functional group; wherein the deoxyribozyme is in an inactive state and wherein R³ contains an aptamer for the ligand so that when the ligand binds to the aptamer, the deoxyribozyme is converted into an active state;
causing the generation of an optical signal by cleavage of the phosphate functional group from the substrate present in the sample by self-phosphorylation of the deoxyribozyme in the active state; and
detecting the generated optical signal.

12. The method according to claim 11, wherein the ligand is selected from an oligonucleotide, a protein, a small molecule, a metal ion, a lipid.

13. The method according to claim 9, wherein the chemiluminescent substrate is a compound selected from the group of phosphate-stabilized 1,2-dioxetanes, preferably selected from disodium 2-chloro-5-(4-methoxyspiro{1,2-dioxetane-3,2′-(5′-chloro)tricyclo[3.3.1.1^{3.7}]decan}-4-yl)phenyl phosphate and disodium 3-(4-methoxyspiro {1,2-dioxetane-3,2′-(5′-chloro)tricyclo[3.3.1.13.7]decan}-4-yl) phenyl phosphate.


SEQUENCE LISTING (242 sequences)

SEQ ID NO: 1 - DNA, 32 bp, artificial
  general sequence of deoxyribozyme
  variation (1)..(1): G or T
  misc_feature (1)..(3): n is a, c, g, t or u
  variation (2)..(2): G or T
  variation (3)..(3): A or C
  variation (5)..(5): G or T
  misc_feature (5)..(18): n is a, c, g, t or u
  variation (6)..(6): A or T
  unsure (7)..(7)
  variation (8)..(8): G or T or A
  variation (9)..(9): A or C or G or T
  variation (10)..(10): A or G or T
  variation (11)..(11): T or C or A or G
  variation (12)..(12): A or T or G
  variation (13)..(13): A or T or G or C
  unsure (14)..(14): helix
  unsure (15)..(15)
  unsure (16)..(16): helix
  variation (17)..(17): T or A or C or G
  variation (18)..(18): G or A
  variation (23)..(23): T or C or G
  misc_feature (23)..(23): n is a, c, g, t or u
  variation (29)..(29): T or G or C
  misc_feature (29)..(32): n is a, c, g, t or u
  unsure (30)..(30): helix
  variation (31)..(31): G or A or C or T
  unsure (32)..(32)
nnnannnnnnnnnnnnnntgacntgggannnn

SEQ ID NO: 2 - DNA, 32 bp, artificial
  general sequence of deoxyribozyme
  variation (3)..(3): A or C
  misc_feature (3)..(3): n is a, c, g, t or u
  variation (6)..(6): A or T
  misc_feature (6)..(7): n is a, c, g, t or u
  unsure (7)..(7)
  variation (11)..(11): T or C
  misc_feature (11)..(11): n is a, c, g, t or u
  variation (13)..(13): A or T or G or C
  misc_feature (13)..(17): n is a, c, g, t or u
  unsure (14)..(14): helix
  unsure (15)..(15)
  unsure (16)..(16): helix
  variation (17)..(17): T or A or C or G
  variation (23)..(23): T or C
  misc_feature (23)..(23): n is a, c, g, t or u
  unsure (30)..(30): helix
  misc_feature (30)..(32): n is a, c, g, t or u
  variation (31)..(31): G or A or C or T
  unsure (32)..(32)
ggnagnngaanannnnngtgacntgggatnnn

SEQ ID NO: 3 - DNA, 32 bp, artificial
  general sequence of deoxyribozyme
  unsure (7)..(7)
  misc_feature (7)..(7): n is a, c, g, t or u
  variation (13)..(13): A or T or G or C
  misc_feature (13)..(17): n is a, c, g, t or u
  unsure (14)..(14): helix
  unsure (15)..(15)
  unsure (16)..(16): helix
  variation (17)..(17): T or A or C or G
  unsure (30)..(30): helix
  misc_feature (30)..(30): n is a, c, g, t or u
  unsure (32)..(32)
  misc_feature (32)..(32): n is a, c, g, t or u
ggaagangaatannnnngtgacttgggatngn

SEQ ID NO: 4 - DNA, 31 bp, artificial
  pool sequence
  unsure (16)..(16): 70 nucleotides
  misc_feature (16)..(16): n is a, c, g, t or u
ggaagagatggcgacnagctgatcctgatgg

SEQ ID NO: 5 - DNA, 15 bp, artificial
  blocking oligonucleotide BO1, REV1
ccatcaggatcagct

SEQ ID NO: 6 - DNA, 23 bp, artificial
  short oligonucleotide, FWD1
gaattctaatacgactcactata

SEQ ID NO: 7 - DNA, 33 bp, artificial
  splint oligonucleotide
gtcgccatctcttcctatagtgagtcgtattag

SEQ ID NO: 8 - DNA, 100 bp, artificial
  deoxyribozyme
ggaagagatggcgacgacacagggacgatgccgaatatcctcagtgcgcagggccgcaggggggagtgacttgggatggggggtcagctgatcctgatgg

SEQ ID NO: 9 - DNA, 105 bp, artificial
  library sequence
ggaagagatggcgacgacacagggacgatgccgaatatcctcagtgcgcagggccgcaggggggagtgacttgggatggggggtccactaatgatctgcccgatg

SEQ ID NO: 10 - DNA, 20 bp, artificial
  blocking oligonucleotide BO2, REV2
catcgggcagatcattagtg

SEQ ID NO: 11 - DNA, 20 bp, artificial
  short oligonucleotide
accgctcaggtgtagtatca

SEQ ID NO: 12 - DNA, 35 bp, artificial
  splint oligonucleotide
gtcgccatctcttcctgatactacacctgagcggt

SEQ ID NO: 13 - DNA, 20 bp, artificial
  FWD2
accgctcaggtgtagtatca

SEQ ID NO: 14 - DNA, 40 bp, artificial
  deoxyribozyme consensus sequence
  unsure (7)..(7): 26 nucleotides
  misc_feature (7)..(7): n is a, c, g, t or u
  unsure (18)..(18): 18 nucleotides
  misc_feature (18)..(18): n is a, c, g, t or u
ggaagangaatatccccnggggagtgacttgggatggggg

SEQ ID NO: 15 - DNA, 105 bp, artificial
  deoxyribozyme
ggaagatatggcgcgaacaaatggacaatgccgaatatcccccgcacgcagggcaacaagggggagtgacttgggatgggggctgcactaatgatctgcccgatg

SEQ ID NO: 16 - DNA, 105 bp, artificial
  deoxyribozyme
ggaagaaatgacgaggccacacggacgaggctgaatatcctcactgcggtgtgccccagggaggagtgacttgggatggtgggttcactaatgatctgcccgatg

SEQ ID NO: 17 - DNA, 105 bp, artificial
  deoxyribozyme
ggaagatagtgtgtcgataaagggacaatgctgaatttcctccctgagccggcgcgtaggggggagtgacttgggatggggggtgcactaatgatctgcccgatg

SEQ ID NO: 18 - DNA, 105 bp, artificial
  deoxyribozyme
ggaagacttgacgagcgcacagcgcttgttcagaatatcacccgtcatgtgttcgtgaagggggagtgacttgggatgggggttccactaatgatctgcccgatg

SEQ ID NO: 19 - DNA, 104 bp, artificial
  deoxyribozyme
ggaagaaatggtgcgacacaggggcgctccagaatatcctcattgcgatatgccgcagggaggagtgacttgggatggtgggtccactaatgatctgcccgatg

SEQ ID NO: 20 - DNA, 105 bp, artificial
  deoxyribozyme
ggaagaaatgtagaggaaacagtgactctgcagaatatcctcactgcgtagtggggcaggggggagtgacttgggatgggggctacactaatgatctgcccgatg

SEQ ID NO: 21 - DNA, 104 bp, artificial
  deoxyribozyme
ggaagaaacgcgacgacacagtaatgatgcggaatatccccccttcatagggcgaaaggggggagtgacttgggatgggggttacactaatgatctgcccgatg

SEQ ID NO: 22 - DNA, 105 bp, artificial
  deoxyribozyme
ggcagagatggcaacgtcaacatgaggatgccgcatatccccagtgcacacttgggcagtggggagtgacttgggatgggggaaacactaatgatctgcccgatg

SEQ ID NO: 23 - DNA, 105 bp, artificial
  deoxyribozyme
ggaagatatggcggtgactcttcgaagctgccgaatatcccccgtgcgcggagcctcaggggggagtgacttgggatggggggtccactaatgatctgcccgatg

SEQ ID NO: 24 - DNA, 105 bp, artificial
  deoxyribozyme
ggaagaaatgccgacctcccagggtggaggctgaatatccaccatggtccggaacggatgggggagtgacttgggatgggggaaccactaatgatctgcccgatg

SEQ ID NO: 25 - DNA, 46 bp, artificial
  minimized catalytic core
ggaagaaaaagaatatccccaaaaggggagtgacttgggatggggg

SEQ ID NO: 26 - DNA, 46 bp, artificial
  deoxyribozyme
ggaagaaaaagaatatcctcaaaaggggagtgacttgggatggggg

SEQ ID NO: 27 - DNA, 46 bp, artificial
  deoxyribozyme
ggaagaaaaagaatatccccaaaagaggagtgacttgggatggggg

SEQ ID NO: 28 - DNA, 46 bp, artificial
  deoxyribozyme
ggaagaaaaagaatatccccaaaaggggagtgacttgggatggtgg

SEQ ID NO: 29 - DNA, 46 bp, artificial
  deoxyribozyme
ggaagaaaaagaatatcctcaaaagaggagtgacttgggatggggg

SEQ ID NO: 30 - DNA, 46 bp, artificial
  deoxyribozyme
ggaagaaaaagaatatcctcaaaaggggagtgacttgggatggtgg

SEQ ID NO: 31 - DNA, 46 bp, artificial
  deoxyribozyme
ggaagaaaaagaatatccccaaaagaggagtgacttgggatggtgg

SEQ ID NO: 32 - DNA, 46 bp, artificial
  deoxyribozyme
ggaagaaaaagaatatcctcaaaagaggagtgacttgggatggtgg

SEQ ID NO: 33 - DNA, 46 bp, artificial
  deoxyribozyme
tgaagaaaaagaatatccccaaaaggggagtgacttgggatggggg

SEQ ID NO: 34 - DNA, 46 bp, artificial
  deoxyribozyme
gtaagaaaaagaatatccccaaaaggggagtgacttgggatggggg

SEQ ID NO: 35 - DNA, 46 bp, artificial
  deoxyribozyme
ggcagaaaaagaatatccccaaaaggggagtgacttgggatggggg

SEQ ID NO: 36 - DNA, 46 bp, artificial
  deoxyribozyme
ggaataaaaagaatatccccaaaaggggagtgacttgggatggggg

SEQ ID NO: 37 - DNA, 46 bp, artificial
  deoxyribozyme
ggaagtaaaagaatatccccaaaaggggagtgacttgggatggggg

SEQ ID NO: 38 - DNA, 46 bp, artificial
  deoxyribozyme
ggaagaaaaataatatccccaaaaggggagtgacttgggatggggg

SEQ ID NO: 39 - DNA, 46 bp, artificial
  deoxyribozyme
ggaagaaaaaaaatatccccaaaaggggagtgacttgggatggggg

SEQ ID NO: 40 - DNA, 46 bp, artificial
  deoxyribozyme
ggaagaaaaagcatatccccaaaaggggagtgacttgggatggggg

SEQ ID NO: 41 - DNA, 46 bp, artificial
  deoxyribozyme
ggaagaaaaaggatatccccaaaaggggagtgacttgggatggggg

SEQ ID NO: 42 - DNA, 46 bp, artificial
  deoxyribozyme
ggaagaaaaagtatatccccaaaaggggagtgacttgggatggggg

SEQ ID NO: 43 - DNA, 46 bp, artificial
  deoxyribozyme
ggaagaaaaagattatccccaaaaggggagtgacttgggatggggg

SEQ ID NO: 44 - DNA, 46 bp, artificial
  deoxyribozyme
ggaagaaaaagagtatccccaaaaggggagtgacttgggatggggg

SEQ ID NO: 45 - DNA, 46 bp, artificial
  deoxyribozyme
ggaagaaaaagaaaatccccaaaaggggagtgacttgggatggggg

SEQ ID NO: 46 - DNA, 46 bp, artificial
  deoxyribozyme
ggaagaaaaagaacatccccaaaaggggagtgacttgggatggggg

SEQ ID NO: 47 - DNA, 46 bp, artificial
  deoxyribozyme
ggaagaaaaagaagatccccaaaaggggagtgacttgggatggggg

SEQ ID NO: 48 - DNA, 46 bp, artificial
  deoxyribozyme
ggaagaaaaagaatttccccaaaaggggagtgacttgggatggggg

SEQ ID NO: 49 - DNA, 46 bp, artificial
  deoxyribozyme
ggaagaaaaagaatgtccccaaaaggggagtgacttgggatggggg

SEQ ID NO: 50 - DNA, 46 bp, artificial
  deoxyribozyme
ggaagaaaaagaatatccccaaaaggggaatgacttgggatggggg

SEQ ID NO: 51 - DNA, 46 bp, artificial
  deoxyribozyme
ggaagaaaaagaatatccccaaaaggggagtgacctgggatggggg

SEQ ID NO: 52 - DNA, 46 bp, artificial
  deoxyribozyme
ggaagaaaaagaatatccccaaaaggggagtgacgtgggatggggg

SEQ ID NO: 53 - DNA, 46 bp, artificial
  deoxyribozyme
ggaagaaaaagaatatccccaaaaggggagtgacttgggagggggg

SEQ ID NO: 54 - DNA, 46 bp, artificial
  deoxyribozyme
ggaagaaaaagaatatccccaaaaggggagtgacttgggacggggg

SEQ ID NO: 55 - DNA, 46 bp, artificial
  deoxyribozyme
ggaagaaaaagaatatccccaaaaggggagtgacttgggatggggt

SEQ ID NO: 56 - DNA, 46 bp, artificial
  deoxyribozyme
ggaagaaaaagaatatccccaaaaggggagtgacttgggatgggga

SEQ ID NO: 57 - DNA, 46 bp, artificial
  deoxyribozyme
ggaagaaaaagaatatccccaaaaggggagtgacttgggatggggc

SEQ ID NO: 58 - DNA, 46 bp, artificial
  deoxyribozyme
ggaagaaaaagaataaccccaaaaggggtgtgacttgggatggggg

SEQ ID NO: 59 - DNA, 46 bp, artificial
  deoxyribozyme
ggaagaaaaagaatacccccaaaaggggggtgacttgggatggggg

SEQ ID NO: 60 - DNA, 46 bp, artificial
  deoxyribozyme
ggaagaaaaagaatagccccaaaaggggcgtgacttgggatggggg

SEQ ID NO: 61 - DNA, 46 bp, artificial
  deoxyribozyme
ggaagaaaaagaatattcccaaaagggaagtgacttgggatagggg

SEQ ID NO: 62 - DNA, 46 bp, artificial
  deoxyribozyme
ggaagaaaaagaatattcccaaaagggaagtgacttgggattgggg

SEQ ID NO: 63 - DNA, 46 bp, artificial
  deoxyribozyme
ggaagaaaaagaatatctccaaaaggagagtgacttgggatgaggg

SEQ ID NO: 64 - DNA, 46 bp, artificial
  deoxyribozyme
ggaagaaaaagaatatctccaaaaggagagtgacttgggatgtggg

SEQ ID NO: 65 - DNA, 46 bp, artificial
  deoxyribozyme
ggaagaaaaagaatatcctcaaaagaggagtgacttgggatggagg

SEQ ID NO: 66 - DNA, 46 bp, artificial; identical to SEQ ID NO: 32
  deoxyribozyme

SEQ ID NO: 67 - DNA, 46 bp, artificial
  deoxyribozyme
ggaagaaaaagaatatccctaaaaagggagtgacttgggatgggag

SEQ ID NO: 68 - DNA, 46 bp, artificial
  deoxyribozyme
ggaagaaaaagaatatccctaaaaagggagtgacttgggatgggtg

SEQ ID NO: 69 - DNA, 46 bp, artificial
  deoxyribozyme
ggaagaaaaagaatatctttaaaaaaagagtgacttgggatgaaag

SEQ ID NO: 70 - DNA, 46 bp, artificial
  deoxyribozyme
ggaagaaaaagaatatctttaaaaaaagagtgacttgggatgtttg

SEQ ID NO: 71 - DNA, 46 bp, artificial
  deoxyribozyme
ggaagaaaaagaatatttttaaaaaaaaagtgacttgggataaaag

SEQ ID NO: 72 - DNA, 46 bp, artificial
  deoxyribozyme
ggaagaaaaagaatatttttaaaaaaaaagtgacttgggatttttg

SEQ ID NO: 73 - DNA, 43 bp, artificial
  deoxyribozyme
ggaagaaaaagaatatcccaaaagggagtgacttgggatgggg

SEQ ID NO: 74 - DNA, 40 bp, artificial
  deoxyribozyme
ggaagaaaaagaatatccaaaaggagtgacttgggatggg

SEQ ID NO: 75 - DNA, 37 bp, artificial
  deoxyribozyme
ggaagaaaaagaatatcaaaagagtgacttgggatgg

SEQ ID NO: 76 - DNA, 46 bp, artificial
  deoxyribozyme
ggaagaaaaagaatatacccaaaaggggagtgacttgggatggggg

SEQ ID NO: 77 - DNA, 46 bp, artificial
  deoxyribozyme
ggaagaaaaagaatatgcccaaaaggggagtgacttgggatggggg

SEQ ID NO: 78 - DNA, 46 bp, artificial
  deoxyribozyme
ggaagaaaaagaatattcccaaaaggggagtgacttgggatggggg

SEQ ID NO: 79 - DNA, 46 bp, artificial
  deoxyribozyme
ggaagaaaaagaatatacccaaaagggaagtgacttgggatagggg

SEQ ID NO: 80 - DNA, 46 bp, artificial
  deoxyribozyme
ggaagaaaaagaatatccccaaaagggaagtgacttgggatagggg

SEQ ID NO: 81 - DNA, 46 bp, artificial
  deoxyribozyme
ggaagaaaaagaatatgcccaaaagggaagtgacttgggatagggg

SEQ ID NO: 82 - DNA, 46 bp, artificial
  deoxyribozyme
ggaagaaaaagaatatacccaaaagggaagtgacttgggattgggg

SEQ ID NO: 83 - DNA, 46 bp, artificial
  deoxyribozyme
ggaagaaaaagaatatccccaaaagggaagtgacttgggattgggg

SEQ ID NO: 84 - DNA, 46 bp, artificial
  deoxyribozyme
ggaagaaaaagaatatgcccaaaagggaagtgacttgggattgggg

SEQ ID NO: 85 - DNA, 46 bp, artificial
  deoxyribozyme
ggaagaaaaagaatatccccaaaaggggagtgacttgggatagggg

SEQ ID NO: 86 - DNA, 46 bp, artificial
  deoxyribozyme
ggaagaaaaagaatatccccaaaaggggagtgacttgggattgggg

SEQ ID NO: 87 - DNA, 46 bp, artificial
  deoxyribozyme
ggaagaaaaagaatattcccaaaagggaagtgacttgggatggggg

SEQ ID NO: 88 - DNA, 46 bp, artificial
  deoxyribozyme
ggaagaaaaagaatattcccaaaaggggagtgacttgggattgggg

SEQ ID NO: 89 - DNA, 46 bp, artificial
  deoxyribozyme
ggaagaaaaagaatatgcccaaaaggggagtgacttgggatcgggg

SEQ ID NO: 90 - DNA, 46 bp, artificial
  deoxyribozyme
ggaagaaaaagaatatccccaaaagggcagtgacttgggatggggg

SEQ ID NO: 91 - DNA, 46 bp, artificial
  deoxyribozyme
ggaagaaaaagaatatccccaaaagggaagtgacttgggatggggg

SEQ ID NO: 92 - DNA, 46 bp, artificial
  deoxyribozyme
ggaagaaaaagaatatcaccaaaaggggagtgacttgggatggggg

SEQ ID NO: 93 - DNA, 46 bp, artificial
  deoxyribozyme
ggaagaaaaagaatatcgccaaaaggggagtgacttgggatggggg

SEQ ID NO: 94 - DNA, 46 bp, artificial
  deoxyribozyme
ggaagaaaaagaatatctccaaaaggggagtgacttgggatggggg

SEQ ID NO: 95 - DNA, 46 bp, artificial
  deoxyribozyme
ggaagaaaaagaatatcaccaaaaggagagtgacttgggatgaggg

SEQ ID NO: 96 - DNA, 46 bp, artificial
  deoxyribozyme
ggaagaaaaagaatatccccaaaaggagagtgacttgggatgaggg

SEQ ID NO: 97 - DNA, 46 bp, artificial
  deoxyribozyme
ggaagaaaaagaatatcgccaaaaggagagtgacttgggatgaggg

SEQ ID NO: 98 - DNA, 46 bp, artificial
  deoxyribozyme
ggaagaaaaagaatatcaccaaaaggagagtgacttgggatgtggg

SEQ ID NO: 99 - DNA, 46 bp, artificial
  deoxyribozyme
ggaagaaaaagaatatccccaaaaggagagtgacttgggatgtggg

SEQ ID NO: 100 - DNA, 46 bp, artificial
  deoxyribozyme
ggaagaaaaagaatatcgccaaaaggagagtgacttgggatgtggg

SEQ ID NO: 101 - DNA, 46 bp, artificial
  deoxyribozyme
ggaagaaaaagaatatccccaaaaggggagtgacttgggatgaggg

SEQ ID NO: 102 - DNA, 46 bp, artificial
  deoxyribozyme
ggaagaaaaagaatatccccaaaaggggagtgacttgggatgtggg

SEQ ID NO: 103 - DNA, 46 bp, artificial
  deoxyribozyme
ggaagaaaaagaatatctccaaaaggagagtgacttgggatggggg

SEQ ID NO: 104 - DNA, 46 bp, artificial
  deoxyribozyme
ggaagaaaaagaatatctccaaaaggggagtgacttgggatgtggg

SEQ ID NO: 105 - DNA, 46 bp, artificial
  deoxyribozyme
ggaagaaaaagaatatcgccaaaaggggagtgacttgggatgcggg

SEQ ID NO: 106 - DNA, 46 bp, artificial
  deoxyribozyme
ggaagaaaaagaatatccccaaaaggcgagtgacttgggatggggg

SEQ ID NO: 107 - DNA, 46 bp, artificial
  deoxyribozyme
ggaagaaaaagaatatccccaaaaggagagtgacttgggatggggg

SEQ ID NO: 108 - DNA, 46 bp, artificial
  deoxyribozyme
ggaagaaaaagaatatccacaaaaggggagtgacttgggatggggg

SEQ ID NO: 109 - DNA, 46 bp, artificial
  deoxyribozyme
ggaagaaaaagaatatccgcaaaaggggagtgacttgggatggggg

SEQ ID NO: 110 - DNA, 46 bp, artificial
  deoxyribozyme
ggaagaaaaagaatatccacaaaagaggagtgacttgggatggagg

SEQ ID NO: 111 - DNA, 46 bp, artificial
  deoxyribozyme
ggaagaaaaagaatatccccaaaagaggagtgacttgggatggagg

SEQ ID NO: 112 - DNA, 46 bp, artificial
  deoxyribozyme
ggaagaaaaagaatatccgcaaaagaggagtgacttgggatggagg

SEQ ID NO: 113 - DNA, 46 bp, artificial
  deoxyribozyme
ggaagaaaaagaatatccacaaaagaggagtgacttgggatggtgg

SEQ ID NO: 114 - DNA, 46 bp, artificial
  deoxyribozyme
ggaagaaaaagaatatccgcaaaagaggagtgacttgggatggtgg

SEQ ID NO: 115 - DNA, 46 bp, artificial
  deoxyribozyme
ggaagaaaaagaatatccccaaaaggggagtgacttgggatggagg

SEQ ID NO: 116 - DNA, 46 bp, artificial
  deoxyribozyme
ggaagaaaaagaatatccgcaaaaggggagtgacttgggatggcgg

SEQ ID NO: 117 - DNA, 46 bp, artificial
  deoxyribozyme
ggaagaaaaagaatatccccaaaagcggagtgacttgggatggggg

SEQ ID NO: 118 - DNA, 46 bp, artificial
  deoxyribozyme
ggaagaaaaagaatatcccaaaaaggggagtgacttgggatggggg

SEQ ID NO: 119 - DNA, 46 bp, artificial
  deoxyribozyme
ggaagaaaaagaatatcccgaaaaggggagtgacttgggatggggg

SEQ ID NO: 120 - DNA, 46 bp, artificial
  deoxyribozyme
ggaagaaaaagaatatccctaaaaggggagtgacttgggatggggg

SEQ ID NO: 121 - DNA, 46 bp, artificial
  deoxyribozyme
ggaagaaaaagaatatcccaaaaaagggagtgacttgggatgggag

SEQ ID NO: 122 - DNA, 46 bp, artificial
  deoxyribozyme
ggaagaaaaagaatatccccaaaaagggagtgacttgggatgggag

SEQ ID NO: 123 - DNA, 46 bp, artificial
  deoxyribozyme
ggaagaaaaagaatatcccgaaaaagggagtgacttgggatgggag

SEQ ID NO: 124 - DNA, 46 bp, artificial
  deoxyribozyme
ggaagaaaaagaatatcccaaaaaagggagtgacttgggatgggtg

SEQ ID NO: 125 - DNA, 46 bp, artificial
  deoxyribozyme
ggaagaaaaagaatatccccaaaaagggagtgacttgggatgggtg

SEQ ID NO: 126 - DNA, 46 bp, artificial
  deoxyribozyme
ggaagaaaaagaatatcccgaaaaagggagtgacttgggatgggtg

SEQ ID NO: 127 - DNA, 46 bp, artificial
  deoxyribozyme
ggaagaaaaagaatatccccaaaaggggagtgacttgggatgggag

SEQ ID NO: 128 - DNA, 46 bp, artificial
  deoxyribozyme
ggaagaaaaagaatatccccaaaaggggagtgacttgggatgggtg

SEQ ID NO: 129 - DNA, 46 bp, artificial
  deoxyribozyme
ggaagaaaaagaatatccctaaaaagggagtgacttgggatggggg

SEQ ID NO: 130 - DNA, 46 bp, artificial
  deoxyribozyme
ggaagaaaaagaatatcccaaaaatgggagtgacttgggatgggag

SEQ ID NO: 131 - DNA, 46 bp, artificial
  deoxyribozyme
ggaagaaaaagaatatcccgaaaacgggagtgacttgggatgggag

SEQ ID NO: 132 - DNA, 46 bp, artificial
  deoxyribozyme
ggaagaaaaagaatatccctaaaaggggagtgacttgggatgggtg

SEQ ID NO: 133 - DNA, 46 bp, artificial
  deoxyribozyme
ggaagaaaaagaatatcccgaaaatgggagtgacttgggatgggag

SEQ ID NO: 134 - DNA, 46 bp, artificial
  deoxyribozyme
ggaagaaaaagaatatcccgaaaaggggagtgacttgggatgggcg

SEQ ID NO: 135 - DNA, 46 bp, artificial
  deoxyribozyme
ggaagaaaaagaatatccccaaaacgggagtgacttgggatggggg

SEQ ID NO: 136 - DNA, 46 bp, artificial
  deoxyribozyme
ggaagaaaaagaatatccccaaaaagggagtgacttgggatggggg

SEQ ID NO: 137 - DNA, 46 bp, artificial
  deoxyribozyme
ggaagaaaaagaatttcctcaaaaggggagtgacttgggatggggg

SEQ ID NO: 138 - DNA, 46 bp, artificial
  deoxyribozyme
ggcagaaaaagcatatccccaaaaggggagtgacttgggatggggg

SEQ ID NO: 139 - DNA, 46 bp, artificial
  deoxyribozyme
ggaagaaaaagcatatcctcaaaaggggagtgacttgggatggggg

SEQ ID NO: 140 - DNA, 46 bp, artificial
  deoxyribozyme
ggaagaaaaagaatatccgcaaaaggggagtgacttgggatggcga

SEQ ID NO: 141 - DNA, 46 bp, artificial
  deoxyribozyme
ggaagaaaaagatagtaaccaaaaggggagtgacttgggatggggg

SEQ ID NO: 142 - DNA, 46 bp, artificial
  deoxyribozyme
ggaagaaaaagcatatccccaaaaggggagtgacttgggatggggt

SEQ ID NO: 143 - DNA, 46 bp, artificial
  deoxyribozyme
ggaagaaaaagaatatcctcaaaaggggagtgacttgggatggggt

SEQ ID NO: 144 - DNA, 46 bp, artificial
  deoxyribozyme
ggaagtaaaagaatatcctcaaaaggggagtgacttgggatggggt

SEQ ID NO: 145 - DNA, 46 bp, artificial
  deoxyribozyme
ggaagaaaaagtatatcctcaaaaggggagtgacttgggatggggg

SEQ ID NO: 146 - DNA, 46 bp, artificial
  deoxyribozyme
ggaagaaaaagcatatccacaaaaggggagtgacttgggatggggg

SEQ ID NO: 147 - DNA, 46 bp, artificial
  deoxyribozyme
ggcagtaaaagcatatccacaaaaggggagtgacttgggatggggg

SEQ ID NO: 148 - DNA, 46 bp, artificial
  deoxyribozyme
ggaagaaaaaggatatcctcaaaaggggagtgacttgggatggggg

SEQ ID NO: 149 - DNA, 46 bp, artificial
  deoxyribozyme
ggaagtaaaagcatatcctcaaaaggggagtgacttgggatggggg

SEQ ID NO: 150 - DNA, 46 bp, artificial
  deoxyribozyme
ggaagaaaaagaatatcatcaaaaggggagtgacttgggatggggg

SEQ ID NO: 151 - DNA, 46 bp, artificial
  deoxyribozyme
ggaagaaaaatatctaccctaaaaggggagtgacttgggatggggg

SEQ ID NO: 152 - DNA, 46 bp, artificial
  deoxyribozyme
ggaagaaaaagcatatccccaaaaggggagtgacttgggatgaggg

SEQ ID NO: 153 - DNA, 46 bp, artificial
  deoxyribozyme
ggaagaaaaagaatatcctcaaaaggggagtgacttgggatagggg

SEQ ID NO: 154 - DNA, 46 bp, artificial
  deoxyribozyme
ggaagaaaaaggatatccacaaaaggggagtgacttgggatggggg

SEQ ID NO: 155 - DNA, 46 bp, artificial
  deoxyribozyme
ggaagaaaaagcatatcctcaaaagaggagtgacttgggatggtgg

SEQ ID NO: 156 - DNA, 46 bp, artificial
  deoxyribozyme
ggcagaaaaagaatatcctcaaaaggggagtgacttgggatggggg

SEQ ID NO: 157 - DNA, 46 bp, artificial
  deoxyribozyme
ggaagtaaaagaatatcaccaaaaggggagtgacttgggatggggg

SEQ ID NO: 158 - DNA, 46 bp, artificial
  deoxyribozyme
ggaagaaaaagaacatcctcaaaaggggagtgacttgggatggggg

SEQ ID NO: 159 - DNA, 46 bp, artificial
  deoxyribozyme
ggaagaaaaagcatatacccaaaaggggagtgacttgggatggggg

SEQ ID NO: 160 - DNA, 46 bp, artificial
  deoxyribozyme
ggaagaaaaagcgtatacccaaaaggggagtgacttgggatggggc

SEQ ID NO: 161 - DNA, 46 bp, artificial
  deoxyribozyme
ggaagtaaaagaatttccccaaaaggggagtgacttgggatggggg

SEQ ID NO: 162 - DNA, 46 bp, artificial
  deoxyribozyme
ggaagaaaaagaatatcctcaaaaggggagtgacttgggatgggga

SEQ ID NO: 163 - DNA, 46 bp, artificial
  deoxyribozyme
ggaagaaaaagaatatccttaaaagaggagtgacttgggatggtgg

SEQ ID NO: 164 - DNA, 46 bp, artificial
  deoxyribozyme
ggcagaaaaagaatatccccaaaaggggaatgacttgggatggggg

SEQ ID NO: 165 - DNA, 46 bp, artificial
  deoxyribozyme
ggaagaaaaagaatattctcaaaaggggagtgacttgggatgaggg

SEQ ID NO: 166 - DNA, 46 bp, artificial
  deoxyribozyme
ggaagaaaaagaatttccccaaaagggaagtgacttgggattgggg

SEQ ID NO: 167 - DNA, 43 bp, artificial
  deoxyribozyme
  unsure (7)..(7): 3 nucleotides
  misc_feature (7)..(7): n is a, c, g, or t
ggaagangaatatccccaaaaggggagtgacttgggatggggg

SEQ ID NO: 168 - DNA, 43 bp, artificial; identical to SEQ ID NO: 167
  deoxyribozyme
  unsure (7)..(7): 10 nucleotides
  misc_feature (7)..(7): n is a, c, g, or t

SEQ ID NO: 169 - DNA, 43 bp, artificial; identical to SEQ ID NO: 167
  deoxyribozyme
  unsure (7)..(7): 30 nucleotides
  misc_feature (7)..(7): n is a, c, g, or t

SEQ ID NO: 170 - DNA, 43 bp, artificial
  deoxyribozyme
  unsure (21)..(21): 3 nucleotides
  misc_feature (21)..(21): n is a, c, g, or t
ggaagaaaaagaatatccccnggggagtgacttgggatggggg

SEQ ID NO: 171 - DNA, 43 bp, artificial; identical to SEQ ID NO: 170
  deoxyribyzme
  unsure (21)..(21): 10 nucleotides
  misc_feature (21)..(21): n is a, c, g, or t

SEQ ID NO: 172 - DNA, 43 bp, artificial; identical to SEQ ID NO: 170
  deoxyribozyme
  unsure (21)..(21): 30 nucleotides
  misc_feature (21)..(21): n is a, c, g, or t

SEQ ID NO: 173 - DNA, 47 bp, artificial
  deoxyribozyme
  unsure (47)..(47): 3 nucleotides
  misc_feature (47)..(47): n is a, c, g, or t
ggaagaaaaagaatatccccaaaaggggagtgacttgggatgggggn

SEQ ID NO: 174 - DNA, 47 bp, artificial; identical to SEQ ID NO: 173
  deoxyribozyme
  unsure (47)..(47): 10 nucleotides
  misc_feature (47)..(47): n is a, c, g, or t

SEQ ID NO: 175 - DNA, 47 bp, artificial; identical to SEQ ID NO: 173
  deoxyribozyme
  unsure (47)..(47): 30 nucleotides
  misc_feature (47)..(47): n is a, c, g, or t

SEQ ID NO: 176 - DNA, 45 bp, artificial
  deoxyribozyme
ggaagaagagaatatccccaaaaggggagtgacttgggatggggg

SEQ ID NO: 177 - DNA, 45 bp, artificial
  deoxyribozyme
ggaagaactgaatatccccaaaaggggagtgacttgggatggggg

SEQ ID NO: 178 - DNA, 45 bp, artificial
  deoxyribozyme
ggaagataagaatatccccaaaaggggagtgacttgggatggggg

SEQ ID NO: 179 - DNA, 45 bp, artificial
  deoxyribozyme
ggaagaaccgaatatccccaaaaggggagtgacttgggatggggg

SEQ ID NO: 180 - DNA, 45 bp, artificial
  deoxyribozyme
ggaagaccagaatatccccaaaaggggagtgacttgggatggggg

SEQ ID NO: 181 - DNA, 52 bp, artificial
  deoxyribozyme
ggaagagggatgaccagaatatccccaaaaggggagtgacttgggatggggg

SEQ ID NO: 182 - DNA, 52 bp, artificial
  deoxyribozyme
ggaagaacgtaatcgggaatatccccaaaaggggagtgacttgggatggggg

SEQ ID NO: 183 - DNA, 52 bp, artificial
  deoxyribozyme
ggaagacctcgctaccgaatatccccaaaaggggagtgacttgggatggggg

SEQ ID NO: 184 - DNA, 52 bp, artificial
  deoxyribozyme
ggaagagggggatcttgaatatccccaaaaggggagtgacttgggatggggg

SEQ ID NO: 185 - DNA, 52 bp, artificial
  deoxyribozyme
ggaagattaacgtctcgaatatccccaaaaggggagtgacttgggatggggg

SEQ ID NO: 186 - DNA, 72 bp, artificial
  deoxyribozyme
ggaagacgcccgctgttttacagtgctgcaaaaaacgaatatccccaaaaggggagtgacttgggatggggg

SEQ ID NO: 187 - DNA, 72 bp, artificial
  deoxyribozyme
ggaagaagtctggccgtcgaatctggcctatgtattgaatatccccaaaaggggagtgacttgggatggggg

SEQ ID NO: 188 - DNA, 72 bp, artificial
  deoxyribozyme
ggaagacataagagtggtggcgaccgttacaattaggaatatccccaaaaggggagtgacttgggatggggg

SEQ ID NO: 189 - DNA, 72 bp, artificial
  deoxyribozyme
ggaagaaatgggctcgaattaacgggtagttaattagaatatccccaaaaggggagtgacttgggatggggg

SEQ ID NO: 190 - DNA, 72 bp, artificial
  deoxyribozyme
ggaagaaggtgataggggagcgcacggaattacccagaatatccccaaaaggggagtgacttgggatggggg

SEQ ID NO: 191 - DNA, 45 bp, artificial
  deoxyribozyme
ggaagaaaaagaatatccccggaggggagtgacttgggatggggg

SEQ ID NO: 192 - DNA, 45 bp, artificial
  deoxyribozyme
ggaagaaaaagaatatccccgagggggagtgacttgggatggggg

SEQ ID NO: 193 - DNA, 45 bp, artificial
  deoxyribozyme
ggaagaaaaagaatatcccctaaggggagtgacttgggatggggg

SEQ ID NO: 194 - DNA, 45 bp, artificial
  deoxyribozyme
ggaagaaaaagaatatccccgctggggagtgacttgggatggggg

SEQ ID NO: 195 - DNA, 45 bp, artificial
  deoxyribozyme
ggaagaaaaagaatatccccaatggggagtgacttgggatggggg

SEQ ID NO: 196 - DNA, 52 bp, artificial
  deoxyribozyme
ggaagaaaaagaatatccccgggaaggcctggggagtgacttgggatggggg

SEQ ID NO: 197 - DNA, 52 bp, artificial
  deoxyribozyme
ggaagaaaaagaatatccccttttactgtgggggagtgacttgggatggggg

SEQ ID NO: 198 - DNA, 52 bp, artificial
  deoxyribozyme
ggaagaaaaagaatatcccccttagcaaatggggagtgacttgggatggggg

SEQ ID NO: 199 - DNA, 52 bp, artificial
  deoxyribozyme
ggaagaaaaagaatatccccaaaaagcatgggggagtgacttgggatggggg

SEQ ID NO: 200 - DNA, 52 bp, artificial
  deoxyribozyme
ggaagaaaaagaatatccccaatttcacccggggagtgacttgggatggggg

SEQ ID NO: 201 - DNA, 72 bp, artificial
  deoxyribozyme
ggaagaaaaagaatatcccctaacgagtctcaaaagctacttcgctataaggggagtgacttgggatggggg

SEQ ID NO: 202 - DNA, 72 bp, artificial
  deoxyribozyme
ggaagaaaaagaatatccccgggtgacaccaccgataacgagccttaaacggggagtgacttgggatggggg

SEQ ID NO: 203 - DNA, 72 bp, artificial
  deoxyribozyme
ggaagaaaaagaatatcccccgaccaccaacccaagtagacattatgagtggggagtgacttgggatggggg

SEQ ID NO: 204 - DNA, 72 bp, artificial
  deoxyribozyme
ggaagaaaaagaatatcccctggctccctccaagggtaggcattcacgagggggagtgacttgggatggggg

SEQ ID NO: 205 - DNA, 72 bp, artificial
  deoxyribozyme
ggaagaaaaagaatatccccgcagtgtagtgagaggacctcagcacacgcggggagtgacttgggatggggg

SEQ ID NO: 206 - DNA, 49 bp, artificial
  deoxyribozyme
ggaagaaaaagaatatccccaaaaggggagtgacttgggatggggggat

SEQ ID NO: 207 - DNA, 49 bp, artificial
  deoxyribozyme
ggaagaaaaagaatatccccaaaaggggagtgacttgggatgggggtgg

SEQ ID NO: 208 - DNA, 49 bp, artificial
  deoxyribozyme
ggaagaaaaagaatatccccaaaaggggagtgacttgggatgggggtgt

SEQ ID NO: 209 - DNA, 49 bp, artificial
  deoxyribozyme
ggaagaaaaagaatatccccaaaaggggagtgacttgggatggggggcg

SEQ ID NO: 210 - DNA, 49 bp, artificial; identical to SEQ ID NO: 207
  deoxyribozyme

SEQ ID NO: 211 - DNA, 56 bp, artificial
  deoxyribozyme
ggaagaaaaagaatatccccaaaaggggagtgacttgggatgggggacctaggcaa

SEQ ID NO: 212 - DNA, 56 bp, artificial
  deoxyribozyme
ggaagaaaaagaatatccccaaaaggggagtgacttgggatgggggtgttcatcaa

SEQ ID NO: 213 - DNA, 56 bp, artificial
  deoxyribozyme
ggaagaaaaagaatatccccaaaaggggagtgacttgggatggggggatagactct

SEQ ID NO: 214 - DNA, 56 bp, artificial
  deoxyribozyme
ggaagaaaaagaatatccccaaaaggggagtgacttgggatgggggatacccttgt

SEQ ID NO: 215 - DNA, 56 bp, artificial
  deoxyribozyme
ggaagaaaaagaatatccccaaaaggggagtgacttgggatgggggcgatctgacg

SEQ ID NO: 216 - DNA, 76 bp, artificial
  deoxyribozyme
ggaagaaaaagaatatccccaaaaggggagtgacttgggatgggggcgaagttggcaaacattataagttggggag

SEQ ID NO: 217 - DNA, 76 bp, artificial
  deoxyribozyme
ggaagaaaaagaatatccccaaaaggggagtgacttgggatgggggcaaggactttatacaatgctcgcgggtcga

SEQ ID NO: 218 - DNA, 76 bp, artificial
  deoxyribozyme
ggaagaaaaagaatatccccaaaaggggagtgacttgggatgggggtggagcatgcattcttggacaaaatcttgt

SEQ ID NO: 219 - DNA, 76 bp, artificial
  deoxyribozyme
ggaagaaaaagaatatccccaaaaggggagtgacttgggatggggggtgctacttaaattctgtgtgtgactttaa

SEQ ID NO: 220 - DNA, 76 bp, artificial
  deoxyribozyme
ggaagaaaaagaatatccccaaaaggggagtgacttgggatgggggttggctgccgtcccaggcggcacgatagac

SEQ ID NO: 221 - DNA, 50 bp, artificial
  deoxyribozyme
ggaagatcctgaatatccccttgaggggagtgacttgggatgggggctag

SEQ ID NO: 222 - DNA, 50 bp, artificial
  deoxyribozyme
ggaagagaacgaatatccccacctggggagtgacttgggatgggggcgaa

SEQ ID NO: 223 - DNA, 50 bp, artificial
  deoxyribozyme
ggaagatgccgaatatccccgtcgggggagtgacttgggatgggggtacg

SEQ ID NO: 224 - DNA, 50 bp, artificial
  deoxyribozyme
ggaagagcgagaatatccccacagggggagtgacttgggatgggggacgg

SEQ ID NO: 225 - DNA, 50 bp, artificial
  deoxyribozyme
ggaagaccctgaatatccccccgtggggagtgacttgggatggggggctc

SEQ ID NO: 226 - DNA, 68 bp, artificial
  deoxyribozyme
ggaagactttacctacgaatatccccgtaggcactcggggagtgacttgggatgggggacgctcccat

SEQ ID NO: 227 - DNA, 68 bp, artificial
  deoxyribozyme
ggaagatcgatgaggcgaatatccccagatcacctaggggagtgacttgggatgggggagcggcaccg

SEQ ID NO: 228 - DNA, 68 bp, artificial
  deoxyribozyme
ggaagactccttttatgaatatccccctgtctttaaggggagtgacttgggatgggggcagatcgcta

SEQ ID NO: 229 - DNA, 68 bp, artificial
  deoxyribozyme
ggaagatcgaccgccagaatatcccctcagcgcgagggggagtgacttgggatgggggagaaggaagt

SEQ ID NO: 230 - DNA, 68 bp, artificial
  deoxyribozyme
ggaagataacgtaggggaatatccccccagaaggtaggggagtgacttgggatgggggccccggacgt

SEQ ID NO: 231 - DNA, 68 bp, artificial
  deoxyribozyme
ggaagaaaaagaatatcccccaatcgtgcggggagtgacttgggatgggggaagcacgattgaccttc

SEQ ID NO: 232 - DNA, 15 bp, artificial
  target
gaaggtcaatcgtgc

SEQ ID NO: 233 - DNA, 68 bp, artificial
  deoxyribozyme
ggaagaaaaagaatatccccctcttaagaggggagtgacttgggatgggggaatcttaagagaaggga

SEQ ID NO: 234 - DNA, 15 bp, artificial
  target
tcccttctcttaaga

SEQ ID NO: 235 - DNA, 68 bp, artificial
  deoxyribozyme
ggaagaaaaagaatatccccggcactgatggggagtgacttgggatgggggaaatcagtgcccagtgg

SEQ ID NO: 236 - DNA, 15 bp, artificial
  target
ccactgggcactgat

SEQ ID NO: 237 - DNA, 68 bp, artificial
  deoxyribozyme
ggaagaaaaagaatatccccatgatcggaggggagtgacttgggatgggggaatccgatcatccgaag

SEQ ID NO: 238 - DNA, 15 bp, artificial
  target
cttcggatgatcgga

SEQ ID NO: 239 - DNA, 68 bp, artificial
  deoxyribozyme
ggaagaaaaagaatatccccagtaatagcggggagtgacttgggatgggggaagctattacttatctt

SEQ ID NO: 240 - DNA, 15 bp, artificial
  target
aagataagtaatagc

SEQ ID NO: 241 - DNA, 12 bp, artificial
  L3 sequence
  variant (1)..(1): G or A
  misc_feature (1)..(1): n is a, c, g, t or u
  variant (6)..(6): T or C or G
  misc_feature (6)..(6): n is a, c, g, t or u
  variant (12)..(12): T or C or G
  misc_feature (12)..(12): n is a, c, g, t or u
ntgacntgggan

SEQ ID NO: 242 - DNA, 12 bp, artificial
  L3 consensus sequence
gtgacttgggat